SEQUENCE LISTING (32 sequences)

SEQ ID NO: 1 - DNA, 8332 bp; single-stranded, linear
gcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagctcaggttatgggaccaatggggcagcccctgcaagtgttgaccctaaatatagaagatgagcatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccttaaaaataaggttaacccgcgaggccccctaatccccttaattcttctgatgctcagaggggtcagtactgcttcgcccggctccagtcctcatcaagtctataatatcacctgggaggtaaccaatggagatcgggagacggtatgggcaacttctggcaaccaccctctgtggacctggtggcctgaccttaccccagatttatgtatgttagcccaccatggaccatcttattgggggctagaatatcaatcccctttttcttctcccccggggcccccttgttgctcagggggcagcagcccaggctgttccagagactgcgaagaacctttaacctccctcacccctcggtgcaacactgcctggaacagactcaagctagaccagacaactcataaatcaaatgagggattttatgtttgccccgggccccaccgcccccgagaatccaagtcatgtgggggtccagactccttctactgtgcctattggggctgtgagacaaccggtagagcttactggaagccctcctcatcatgggatttcatcacagtaaacaacaatctcacctctgaccaggctgtccaggtatgcaaagataataagtggtgcaaccccttagttattcggtttacagacgccgggagacgggttacttcctggaccacaggacattactggggcttacgtttgtatgtctccggacaagatccagggcttacatttgggatccgactcagataccaaaatctaggaccccgcgtcccaatagggccaaaccccgttctggcagaccaacagccactctccaagcccaaacctgttaagtcgccttcagtcaccaaaccacccagtgggactcctctctcccctacccaacttccaccggcgggaacggaaaataggctgctaaacttagtagacggagcctaccaagccctcaacctcaccagtcctgacaaaacccaagagtgctggttgtgtctagtagcgggacccccctactacgaaggggttgccgtcctgggtacctactccaaccatacctctgctccagccaactgctccgtggcctcccaacacaagttgaccctgtccgaagtgaccggacagggactctgcataggagcagttcccaaaacacatcaggccctatgtaataccacccagacaagcagtcgagggtcctattatctagttgcccctacaggtaccatgtgggcttgtagtaccgggcttactccatgcatctccaccaccatactgaaccttaccactgattattgtgttcttgtcgaactctggccaagagtcacctatcattcccccagctatgtttacggcctgtttgagagatccaaccgacacaaaagagaaccggtgtcgttaaccctggccctattattgggtggactaaccatggggggaattgccgctggaataggaacagggactactgctctaatggccactcagcaattccagcagctccaagccgcagtacaggatgatctcagggaggttgaaaaatcaatctctaacctagaaaagtctctcacttccctgtctgaagttgtcctacagaatcgaaggggcctagacttgttatttctaaaagaaggagggctgtgtgctgctctaaaagaagaatgttgcttctatgcggaccacacaggactagtgagagacagcatggccaaattgagagagaggcttaatcagagacagaaactgtttgagtcaactcaaggatggtttgagggactgtttaacagatccccttggtttaccaccttgatatctaccattatgggacccctcattgtactcctaatgattttgctcttcggaccctgcattcttaatcgattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgca

SEQ ID NO: 2 - DNA, 36 bp; single-stranded, linear
ggtaacagtctggcccgaattctcagacaaatacag

SEQ ID NO: 3 - DNA, 38 bp; single-stranded, linear
ctgtatttgtctgagaattaaggctagactgttaccac

SEQ ID NO: 4 - DNA, 37 bp; single-stranded, linear
tcgaggatcgcgccgggcggccgcatcgatgtcgacg

SEQ ID NO: 5 - DNA, 35 bp; single-stranded, linear
cgcgtcgacatcgatgcggccgcccgggcggatcc

SEQ ID NO: 6 - DNA, 35 bp; single-stranded, linear
ggggggctcgagatgacgcgcggcttgcgcaatgc

SEQ ID NO: 7 - DNA, 26 bp; single-stranded, linear
ggggggatcgatttcatcatcataca

SEQ ID NO: 8 - DNA, 29 bp; single-stranded, linear
ggggggctcgagggcaccatggagctcag

SEQ ID NO: 9 - DNA, 30 bp; single-stranded, linear
ggggggatcgatccctcagaagctggtgtg

SEQ ID NO: 10 - DNA, 31 bp; single-stranded, linear
ccccccctcgagacctgtcccccccatggag

SEQ ID NO: 11 - DNA, 31 bp; single-stranded, linear
ccccccatcgatgtgggctcacagagggcgc

SEQ ID NO: 12 - DNA, 35 bp; single-stranded, linear
ataaaggcggtcctttcagggggcagccccttcta

SEQ ID NO: 13 - DNA, 35 bp; single-stranded, linear
ggggctgccccctgaaaggaccgcctttatcaaag

SEQ ID NO: 14 - DNA, 33 bp; single-stranded, linear
ggggggctcgagccccgacaccgcggcgggccg

SEQ ID NO: 15 - DNA, 30 bp; single-stranded, linear
ggggggatcgatgctgaagtatctggaacc

SEQ ID NO: 16 - DNA, 30 bp; single-stranded, linear
ggggggctcgagaacaacatcaccatgaca

SEQ ID NO: 17 - DNA, 30 bp; single-stranded, linear
ggggggatcgattcanacdatngcyctraa

SEQ ID NO: 18 - DNA, 37 bp; single-stranded, linear
tcgaggatccgcccgggcggccgcatcgatgtcgacg

SEQ ID NO: 19 - DNA, 35 bp; single-stranded, linear; identical to SEQ ID NO: 5

SEQ ID NO: 20 - DNA, 2409 bp; single-stranded, linear
  CDS 61..2091
gaattcgggcgcgaagcggccggcctgggcgccgactgcagagccgggaggctggtggtcatgccggggttcctggttcgcatcctccttctgctgctggttctgctgcttctgggccctacgcgcggcttgcgcaatgccacccagaggatgtttgaaattgactatagccgggactccttcctcaaggatggccagccatttcgctacatctcaggaagcattcactactcccgtgtgccccgcttctactggaaggaccggctgctgaagatgaagatggctgggctgaacgccatccagacgtatgtgccctggaactttcatgagccctggccaggacagtaccagttttctgaggaccatgatgtggaatattttcttcggctggctcatgagctgggactgctggttatcctgaggcccgggccctacatctgtgcagagtgggaaatgggaggattacctgcttggctgctagagaaagagtctattcttctccgctcctccgacccagattacctggcagctgtggacaagtggttgggagtccttctgcccaagatgaagcctctcctctatcagaatggagggccagttataacagtgcaggttgaaaatgaatatggcagctactttgcctgtgattttgactacctgcgcttcctgcagaagcgctttcgccaccatctgggggatgatgtggttctgtttaccactgatggagcacataaaacattcctgaaatgtggggccctgcagggcctctacaccacggtggactttggaacaggcagcaacatcacagatgctttcctaagccagaggaagtgtgagcccaaaggacccttgatcaattctgaattctatactggctggctagatcactggggccaacctcactccacaatcaagaccgaagcagtggcttcctccctctatgatatacttgcccgtggggcgagtgtgaacttgtacatgtttataggtgggaccaattttgcctattggaatggggccaactcaccctatgcagcacagcccaccagctacgactatgatgccccactgagtgaggctggggacctcactgagaagtattttgctctgcgaaacatcatccagaagtttgaaaaagtaccagaaggtcctatccctccatctacaccaaagtttgcatatggaaaggtcactttggaaaagttaaagacagtgggagcagctctggacattctgtgtccctctgggcccatcaaaagcctttatcccttgacatttatccaggtgaaacagcattatgggtttgtgctgtaccggacaacacttcctcaagattgcagcaacccagcacctctctcttcacccctcaatggagtccacgatcgagcatatgttgctgtggatgggatcccccagggagtccttgagcgaaacaatgtgatcactctgaacataacagggaaagctggagccactctggaccttctggtagagaacatgggacgtgtgaactatggtgcatatatcaacgattttaagggtttggtttctaacctgactctcagttccaatatcctcacggactggacgatctttccactggacactgaggatgcagtgcgcagccacctggggggctggggacaccgtgacagtggccaccatgatgaagcctgggcccacaactcatccaactacacgctcccggccttttatatggggaacttctccattcccagtgggatcccagacttgccccaggacacctttatccagtttcctggatggaccaagggccaggtctggattaatggctttaaccttggccgctattggccagcccggggccctcagttgaccttgtttgtgccccagcacatcctgatgacctcggccccaaacaccatcaccgtgctggaactggagtgggcaccctgcagcagtgatgatccagaactatgtgctgtgacgttcgtggacaggccagttattggctcatctgtgacctacgatcatccctccaaacctgttgaaaaaagactcatgcccccacccccgcaaaaaaacaaagattcatggctggaccatgtatgatgatgaaagcctgtgtctttgagggattctaccctgaacatacctcacagatcctccctgtcatgccacatttcactgattggaatgtggaaatggaaaaggaatttaggatgtgcattttcacctgaggtttccctgcatccctgcagtgccaaagccccaccttcagggaccacctggaatgtgtgaggggctgacagcacagtaacgtgcatacatatctgcagggctggaatggaagctttaaaggtggtagtgatttttattttggaagaatcatgttacctttttgttaaataaaatttgcccgaattc

SEQ ID NO: 21 - protein, 677 aa
MPGFLVRILLLLLVLLLLGPTRGLRNATQRMFEIDYSRDSFLKDGQPFRYISGSIHYSRVPRFYWKDRLLKMKMAGLNAIQTYVPWNFHEPWPGQYQFSEDHDVEYFLRLAHELGLLVILRPGPYICAEWEMGGLPAWLLEKESILLRSSDPDYLAAVDKWLGVLLPKMKPLLYQNGGPVITVQVENEYGSYFACDFDYLRFLQKRFRHHLGDDVVLFTTDGAHKTFLKCGALQGLYTTVDFGTGSNITDAFLSQRKCEPKGPLINSEFYTGWLDHWGQPHSTIKTEAVASSLYDILARGASVNLYMFIGGTNFAYWNGANSPYAAQPTSYDYDAPLSEAGDLTEKYFALRNIIQKFEKVPEGPIPPSTPKFAYGKVTLEKLKTVGAALDILCPSGPIKSLYPLTFIQVKQHYGFVLYRTTLPQDCSNPAPLSSPLNGVHDRAYVAVDGIPQGVLERNNVITLNITGKAGATLDLLVENMGRVNYGAYINDFKGLVSNLTLSSNILTDWTIFPLDTEDAVRSHLGGWGHRDSGHHDEAWAHNSSNYTLPAFYMGNFSIPSGIPDLPQDTFIQFPGWTKGQVWINGFNLGRYWPARGPQLTLFVPQHILMTSAPNTITVLELEWAPCSSDDPELCAVTFVDRPVIGSSVTYDHPSKPVEKRLMPPPPQKNKDSWLDHV

SEQ ID NO: 22 - DNA, 1634 bp; single-stranded, linear
  CDS 13..1617
tgccctccagacatgctggggccctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcccatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccggggcggtccgtggtccccgcgttgcttcctctgctggccgggaccctgctgctgctggagacggccactgctccctgagtgtcccgtccctg

SEQ ID NO: 23 - protein, 535 aa [sequence omitted]

SEQ ID NO: 24 - DNA, 1143 bp; single-stranded, linear
  CDS 7..1137
ggaaccatggagctcagcgtcctcctcttccttgcactcctcacaggcctcttgctactcctggttcagcgtcaccctaactcccatggcaccctcccaccagggccccgccctctgccccttttggggaaccttctgcagatggacagaagaggcctactcaaatcctttctgaggttccgagagaaatatggggacgtcttcacggtacacctgggaccgaggcccgtggtcatgctgtgtggagtagaggccatacgggaggccctggtggacaacgctgaggccttctctggccggggaaaaatcgtcatcatggacccagtctaccagggatatggcatgctctttgccaatggaaaccgctggaaggtgcttcggcgattctctgtgaccaccatgagggacttcgggatgggaaagcggagtgtggaggagcggattcaggacgaggctcagtgtctgatagaggaacttcggaaatccaagggagccctcgtggaccccaccttcctcttccattccattaccgccaacatcatctgctccatcatctttggaaaacgcttccactaccaagatcaagagttcctgaagacgctgaacttgttctgccagagtttcttactcatcagctctatatccagccagctgtttgagctcttctctggcttcttgaaatactttcctggggcacacaggcaagtttacaaaaacctacaggaaatcaatgcttacattggccacagtgtggagaagcaccgtgaaaccctggaccccagcgcccccagggacctcatcgacacctacctgctccacatggaaaaagagaaatccaacccacacagtgaattcagccaccagaacctcatcatcaacacgctctcgctcttctttgctggcactgagaccaccagcaccactctccgctacggcttcctgctcatgctcaaataccctcatgtcgcagagagagtctacaaggagattgaacaggtggttggcccacatcgccctccagcgcttgatgaccgagccaaaatgccatacacagaggcagtcatccgtgagattcagagatttgctgaccttctccccatgggtgtgccccacattgtcacccaacacaccagcttctgaggg

SEQ ID NO: 25 - protein, 377 aa
MELSVLLFLALLTGLLLLLVQRHPNSHGTLPPGPRPLPLLGNLLQMDRRGLLKSFLRFREKYGDVFTVHLGPRPVVMLCGVEAIREALVDNAEAFSGRGKIVIMDPVYQGYGMLFANGNRWKVLRRFSVTTMRDFGMGKRSVEERIQDEAQCLIEELRKSKGALVDPTFLFHSITANIICSIIFGKRFHYQDQEFLKTLNLFCQSFLLISSISSQLFELFSGFLKYFPGAHRQVYKNLQEINAYIGHSVEKHRETLDPSAPRDLIDTYLLHMEKEKSNPHSEFSHQNLIINTLSLFFAGTETTSTTLRYGFLLMLKYPHVAERVYKEIEQVVGPHRPPALDDRAKMPYTEAVIREIQRFADLLPMGVPHIVTQHTSF

SEQ ID NO: 26 - DNA, 2410 bp; single-stranded, linear
  CDS 17..2398
gccacctgtcccccccatggagctgaggccctggttgctatgggtggtagcagcaacaggaaccttggtcctgctagcagctgatgctcagggccagaaggtcttcaccaacacgtgggctgtgcgcatccctggaggcccagcggtggccaacagtgtggcacggaagcatgggttcctcaacctgggccagatcttcggggactattaccacttctggcatcgaggagtgacgaagcggtccctgtcgcctcaccgcccgcggcacagccggctgcagagggagcctcaagtacagtggctggaacagcaggtggcaaagcgacggactaaacgggacgtgtaccaggagcccacagaccccaagtttcctcagcagtggtacctgtctggtgtcactcagcgggacctgaatgtgaaggcggcctgggcgcagggctacacagggcacggcattgtggtctccattctggacgatggcatcgagaagaaccacccggacttggcaggcaattatgatcctggggccagttttgatgtcaatgaccaggaccctgacccccagcctcggtacacacagatgaatgacaacaggcacggcacacggtgtgcgggggaagtggctgcggtggccaacaacggtgtctgtggtgtaggtgtggcctacaacgcccgcattggaggggtgcgcatgctggatggcgaggtgacagatgcagtggaggcacgctcgctgggcctgaaccccaaccacatccacatctacagtgccagctggggccccgaggatgacggcaagacagtggatgggccagcccgcctcgccgaggaggccttcttccgtggggttagccagggccgaggggggctgggctccatctttgtctgggcctcggggaacgggggccgggaacatgacagctgcaactgcgacggctacaccaacagtatctacacgctgtccatcagcagcgccacgcagtttggcaacgtgccgtggtacagcgaggcctgctcgtccacactggccacgacctacagcagtggcaaccagaatgagaagcagatcgtgacgactgacttgcggcagaagtgcacggagtctcacacgggcacctcagcctctgcccccttagcagccggcatcattgctctcaccctggaggccaataagaacctcacatggcgggacatgcaacacctggtggtacagacctcgaagccagcccacctcaatgccaacgactgggccaccaatggtgtgggccggaaagtgagccactcatatggctacgggcttttggacgcaggcgccatggtggccctggcccagaattggaccacagtggccccccagcggaagtgcatcatcgacatcctcaccgagcccaaagacatcgggaaacggctcgaggtgcggaagaccgtgaccgcgtgcctgggcgagcccaaccacatcactcggctggagcacgctcaggcgcggctcaccctgtcctataatcgccgtggcgacctggccatccacctggtcagccccatgggcacccgctccaccctgctggcagccaggccacatgactactccgcagatgggtttaatgactgggccttcatgacaactcattcctgggatgaggatccctctggcgagtgggtcctagagattgaaaacaccagcgaagccaacaactatgggacgctgaccaagttcaccctcgtactctatggcaccgcccctgaggggctgcccgtacctccagaaagcagtggctgcaagaccctcacgtccagtcaggcctgtgtggtgtgcgaggaaggcttctccctgcaccagaagagctgtgtccagcactgccctccaggcttcgccccccaagtcctcgatacgcactatagcaccgagaatgacgtggagaccatccgggccagcgtctgcgccccctgccacgcctcatgtgccacatgccaggggccggccctgacagactgcctcagctgccccagccacgcctccttggaccctgtggagcagacttgctcccggcaaagccagagcagccgagagtccccgccacagcagcagccacctcggctgcccccggaggtggaggcggggcaacggctgcgggcagggctgctgccctcacacctgcctgaggtggtggccggcctcagctgcgccttcatcgtgctggtcttcgtcactgtcttcctggtcctgcagctgcgctctggctttagttttcggggggtgaaggtgtacaccatggaccgtggcctcatctcctacaaggggctgccccctgaagcctggcaggaggagtgcccgtctgactcagaagaggacgagggccggggcgagaggaccgcctttatcaaagaccagagcgccctctgatgagcccac

SEQ ID NO: 27 - protein, 794 aa
MELRPWLLWVVAATGTLVLLAADAQGQKVFTNTWAVRIPGGPAVANSVARKHGFLNLGQIFGDYYHFWHRGVTKRSLSPHRPRHSRLQREPQVQWLEQQVAKRRTKRDVYQEPTDPKFPQQWYLSGVTQRDLNVKAAWAQGYTGHGIVVSILDDGIEKNHPDLAGNYDPGASFDVNDQDPDPQPRYTQMNDNRHGTRCAGEVAAVANNGVCGVGVAYNARIGGVRMLDGEVTDAVEARSLGLNPNHIHIYSASWGPEDDGKTVDGPARLAEEAFFRGVSQGRGGLGSIFVWASGNGGREHDSCNCDGYTNSIYTLSISSATQFGNVPWYSEACSSTLATTYSSGNQNEKQIVTTDLRQKCTESHTGTSASAPLAAGIIALTLEANKNLTWRDMQHLVVQTSKPAHLNANDWATNGVGRKVSHSYGYGLLDAGAMVALAQNWTTVAPQRKCIIDILTEPKDIGKRLEVRKTVTACLGEPNHITRLEHAQARLTLSYNRRGDLAIHLVSPMGTRSTLLAARPHDYSADGFNDWAFMTTHSWDEDPSGEWVLEIENTSEANNYGTLTKFTLVLYGTAPEGLPVPPESSGCKTLTSSQACVVCEEGFSLHQKSCVQHCPPGFAPQVLDTHYSTENDVETIRASVCAPCHASCATCQGPALTDCLSCPSHASLDPVEQTCSRQSQSSRESPPQQQPPRLPPEVEAGQRLRAGLLPSHLPEVVAGLSCAFIVLVFVTVFLVLQLRSGFSFRGVKVYTMDRGLISYKGLPPEAWQEECPSDSEEDEGRGERTAFIKDQSAL

SEQ ID NO: 28 - protein, 4 aa
RKKR

SEQ ID NO: 29 - DNA, 21 bp; single-stranded, linear
ctgtaggtttggcaagctagc

SEQ ID NO: 30 - DNA, 19 bp; single-stranded, linear
cgctgacgggtagtcaatc

SEQ ID NO: 31 - DNA, 27 bp; single-stranded, linear
gaatggcaaagtaaaggtttttcaggg

SEQ ID NO: 32 - DNA, 44 bp; single-stranded, linear
atagttagcggccgcaacccgggccaccctcagtagaggtcctg

(19) United States
(12) Patent Application Publication
(10) Pub. No.: US 2002/0082224 A1
(43) Pub. Date: Jun. 27, 2002

(54) NON-IMMUNOGENIC PRODRUGS AND SELECTABLE MARKERS FOR USE IN GENE THERAPY

(76) Inventors: DOUGLAS J. JOLLY, LEUCADIA, CA (US); MARGARET D. MOORE, SAN DIEGO, CA (US); SUNIL CHADA, VISTA, CA (US)

Correspondence Address: Chiron Corporation, Intellectual Property - R440, P.O. Box 8097, Emeryville, CA, 94662-8097, US

(21) Appl. No.: 09/006,298

(22) Filed: Jan. 13, 1998

Related U.S. Application Data
(60) Provisional application No. 60/035,473, filed on Jan. 14, 1997.
(60) Provisional application No. 60/038,339, filed on Feb. 27, 1997.

(51) Int. Cl. A61K 48/00

(52) U.S. Cl. 514/44; 424/93.1; 435/320.1

(57) ABSTRACT

The present invention provides methods for delivering a gene delivery vehicle to a warm-blooded animal, comprising the step of administering to a warm-blooded animal a gene delivery vehicle which directs the expression of a non-immunogenic selectable marker. Within other aspects, methods are provided for delivering a gene delivery vehicle to a warm-blooded animal, comprising the step of administering to a warm-blooded animal a gene delivery vehicle which directs the expression of a non-immunogenic molecule which is capable of activating an otherwise inactive compound into an active compound.

### CROSS-REFERENCE TO RELATED APPLICATIONS

This application claims the benefit of U.S. Provisional Application No. 60/035,473, filed Jan. 14, 1997, and U.S. Provisional Application No. 60/038,339, filed Feb. 27, 1997, which applications are incorporated by reference in their entirety.

### TECHNICAL FIELD

The present invention relates generally to nucleic acid vectors, and more specifically, to vectors which are capable of delivering a gene of interest to susceptible target cells. These vector constructs are designed to deliver a non-immunogenic gene product which is capable of activating a compound with little or no activity into an active product.

### BACKGROUND OF THE INVENTION

Human gene therapy is a clinical strategy wherein the genetic repertoire of cells are altered either to gain an understanding of the cell's function, or for therapeutic benefit. Briefly, gene therapy involves delivering vectors (e.g., a retrovirus, adenovirus, vaccinia virus, or naked DNA alone) to cells so that therapeutically beneficial genetic information that is contained within the vector can be transferred from the vectors to the cells. This strategy has now been widely applied, with clinical trials presently ongoing for a wide range of both hereditary (e.g., ADA deficiency, familial hypercholesterolemia, and cystic fibrosis) and acquired (e.g., tumors) diseases (Crystal Science 270:404-410, 1995).

It is now clear, however, that long-term expression of foreign genes introduced by gene therapy may lead to immune responses in patients that destroy the treated cells (e.g., C. Bordignon, Brit. J Hematology 93-S2:306, 1996; S. R. Riddell et al., Nature Medicine 2:216, 1996). Although the actual tissue transduced may affect this and although many means of avoiding or minimizing this have been proposed (see J. D. Davies et al., J. Immunol. 157:529, 1996; D. J. Lenschow et al., Science 257, 789, 1992; A. Waisman et al., Nature Medicine 2:889, 1996), it is clear that this limitation is not easily overcome. Moreover, this problem extends to genes that are included in gene transfer vectors (e.g., the neomycin resistance gene) that are included for ease of handling, testing, characterization and manufacturing of gene delivery vehicles. It also extends to the use of genes that are included either to ablate a tumor or tissue or as a “fail-safe” mechanism so that cells that have been treated by genes or in some other way can be destroyed. An archetype of this is the HSV-TK gene.

The present invention provides novel compositions and methods for treating a variety of diseases (e.g., viral diseases, cancer, genetic diseases and others) that overcome previous difficulties associated with the use of vectors in gene therapy, and further provides other, related advantages.

### SUMMARY OF THE INVENTION

Briefly stated, the present invention provides recombinant gene delivery vehicles and methods of using such vehicles for the treatment of a wide variety of pathogenic agents. In particular, utilizing the vectors provided herein one can avoid problems in treating human patients through the use of human genes for selectable markers or activation of prodrugs. The selectable marker can allow biochemical selection (e.g, hypoxanthine phosphoribosyltransferase) color selection (e.g., alkaline phosphatase or beta galactosidase) or selection by antibody binding (e.g., membrane bound alkaline phosphatase, CD 34). The activation of prodrugs can be of various pyrimidine or purine analogues (e.g., deoxycytidine kinase and cytosine arabinoside), other prodrugs from the cancer field. (See for example A. K. Sinhabubu and D. R. Thakker, Advanced Drug Delivery Reviews 19:241, 1996 and M. A. Graham et al., Pharmac. Ther. 51:275, 1991 (both incorporated by reference) such as alkaline phosphatase acid phosphatase, beta-glucuronidase, carboxypeptidase A, cytosine deaminase, nitroreductase (a.k.a. azoredactase or DT diaphorase) plasmin and γ-glutamyl transpeptidase.

Within one aspect of the present invention, methods are provided delivering a gene delivery vehicle to a warm-blooded animal, comprising administering to a warm-blooded animal a gene delivery vehicle which directs the expression of a non-immunogenic selectable marker. Within other related aspects methods are provided for delivering a gene delivery vehicle to a warm-blooded animal, comprising administering to a warm-blooded animal a gene delivery vehicle which directs the expression of a non-immunogenic molecule which is capable of activating an otherwise inactive compound into an active compound. Within one embodiment, the non-immunogenic molecule is selected from the group consisting of alkaline phosphatase, α-Galactosidase, β-glucosidase, β-glucuronidase, Carboxypeptidase A, Cytochrome P450, γ-glutamyl transferase; reductases such as Azoreductase, DT diaphorase and Nitroreductase; and oxidases such as glucose oxidase and xanthine oxidase.

Within other aspects of the invention, gene delivery vehicles are provided which direct the expression of a protein that is toxic upon processing or modification by a protein derived from a pathogenic agent. Within one embodiment, the protein which is toxic upon processing or modification is proricin.

Within yet certain embodiments of the invention, gene delivery vehicles are provided carrying a vector construct comprising a cytotoxic gene under the transcriptional control of an event-specific promoter, such that upon activation of the event-specific promoter the cytotoxic gene is expressed. Within various embodiments, the event-specific promoter is a cellular thymidine kinase promoter, or a thymidylate synthase promoter. Within another embodiment, the event-specific promoter is activated by a hormone. Within yet other embodiments, the cytotoxic gene is selected from the group consisting of ricin, abrin, diphtheria toxin, cholera toxin, gelonin, pokeweed, antiviral protein, tritin, Shigella toxin, and Pseudomonas exotoxin A.

Within another embodiments of the present invention, gene delivery vehicles are provided comprising a cytotoxic gene under the transcriptional control of a tissue-specific promoter (including tissue-specific elements, such as for example, a locus control region), such that upon activation of the tissue-specific promoter the cytotoxic gene is expressed. Within various embodiments, the tissue-specific promoter is the PEPCK promoter, HER2/neu promoter, casein promoter, IgG promoter, Chorionic Embryonic Antigen promoter, elastase promoter, porphobilinogen deaminase promoter, insulin promoter, growth hormone factor promoter, tyrosine hydroxylase promoter, albumin promoter, alphafetoprotein promoter, acetyl-choline receptor promoter, alcohol dehydrogenase promoter, α or β globin promoter, T-cell receptor promoter (including the CD2 LCR), the osteocalcin promoter the IL-2 promoter, IL-2 receptor promoter, whey (wap) promoter, and the MHC Class II promoter.

Within yet another embodiment of the present invention, gene delivery vehicles are provided comprising a cytotoxic gene under the transcriptional control of both an event-specific promoter and a tissue-specific promoter, such that the cytotoxic gene is maximally expressed only upon activation of both the event-specific promoter and the tissue-specific promoter. Representative event-specific and tissue-specific promoters have been discussed above. Within one preferred embodiment, the event-specific promoter is thymidine kinase, and the tissue-specific promoter is selected from the group consisting of the casein promoter and the HER2/neu promoter.

Within other embodiments of the present invention, the gene delivery vehicles described herein may also direct the expression of additional non-vector derived genes (i.e., a heterologous nucleic acid sequence). Within one embodiment the heterologous nucleic acid sequence encodes a protein, such as an immune accessory molecule. Representative examples of immune accessory molecules include IL-1, IL-2, IL-3, IL-4, IL-5, IL-6, IL-7, IL-8, IL-9, IL-10, IL-11, IL-12, IL-13, IL-14, IL-15, B7, B7-2, GM-CSF, CD3, ICAM-1, ICAM-2, β-microglobulin, LFA-3, HLA Class I, and HLA Class II molecules. Within one preferred embodiment, the protein is gamma-interferon.

Within other embodiments, the gene delivery vehicle may also direct the expression of an antisense sequence or ribozyme. Within further embodiments, the gene delivery vehicle may direct the expression of a replacement gene such as Factor VIII, glucocerebrosidase, FIX, ADA, HPRT, CFTCR or the LDL Receptor. Within yet other embodiments, the gene delivery vehicle may direct the expression of a disease associated antigen, such as an immunogenic portion of a virus selected from the group consisting of HBV, HCV, HPV, EBV, FeLV, FIV and HIV.

These and other aspects of the present invention will become evident upon reference to the following detailed description and attached drawings. In addition, various references are set forth below which describe in more detail certain procedures or compositions (e.g., plasmids, etc.), and are therefore incorporated by reference in their entirety.

### BRIEF DESCRIPTION OF THE DRAWINGS

FIG. 1 is a schematic illustration of KT-1.

FIG. 2 is a schematic illustration of KS2+Eco571-LTR(+).

FIG. 3 is a schematic illustration of BA5.

FIG. 4 is a schematic illustration of pBa6B-L 1.

FIGS. 5A and 5B depict a sequence of human beta galactosidase (SEQ ID NOS: 20 and 21).

FIG. 6 is a schematic illustration of pKT/βGal.

FIGS. 7A and 7B depict a sequence of human placental alkaline phosphatase (SEQ ID NOS: 22 and 23).

FIG. 8 is a schematic illustration of pMGA/PLAP.

FIG. 9 is a sequence of human cytochrome P-450 2B (CYP2B) (SEQ ID NOS: 24 and 25).

FIG. 10 is a schematic illustration of pBA6B/CYP2A.

FIGS. 11 A and 11 B depict a sequence of human furin cDNA (SEQ ID NOS: 26 and 27).

FIG. 12 is a schematic illustration of pBA6B/Xfur.

### DETAILED DESCRIPTION OF THE INVENTION

Prior to setting forth the invention, it may be helpful to an understanding thereof to first set forth definitions of certain terms that will be used hereinafter.

“Gene delivery vehicle” refers to a construct which is capable of delivering, and, within preferred embodiments expressing, one or more gene(s) or sequence(s) of interest in a host cell. Representative examples of such vehicles include viral vectors, nucleic acid expression vectors in combination with facilatating agents such as liposomes or polycation condensing agents, naked DNA, and certain eukaryotic cells (e.g., producer cells). Within particularly preferred embodiments of the invention, the gene delivery vehicle includes a member of the high affinity binding pair (discussed below), either expressed on, or included as, an integral part of the exterior of the gene delivery vehicle.

“Retroviral vector construct” refers to an assembly which is, within preferred embodiments of the invention, capable of directing the expression of a sequence(s) or gene(s) of interest. Preferably, the retroviral vector construct should include a 5′ LTR, a tRNA binding site, a packaging signal, one or more heterologous sequences, an origin of second strand DNA synthesis and a 3′ LTR. A wide variety of heterologous sequences may be included within the vector construct, including for example, sequences which encode a protein (e.g., cytotoxic protein, disease-associated antigen, immune accessory molecule, or replacement protein), or which are useful as a molecule itself (e.g., as a ribozyme or antisense sequence). Alternatively, the heterologous sequence may merely be a “stuffer” or “filler” sequence, which is of a size sufficient to allow production of viral particles containing the RNA genome. Preferably, the heterologous sequence is at least 1, 2, 3, 4, 5, 6, 7 or 8 kB in length.

The retroviral vector construct may also include transcriptional promoter/enhancer or locus defining element(s), or other elements which control gene expression by means such as alternate splicing, nuclear RNA export, post-translational modification of messenger, or post-transcriptional modification of protein. Optionally, the retroviral vector construct may also include non-immunogenic selectable markers such as described in this application, as well as one or more specific restriction sites and a translation termination sequence.

“Nucleic Acid Expression Vector” refers to an assembly which is capable of directing the expression of a sequence or gene of interest. The nucleic acid expression vector must include a promoter which, when transcribed, is operably linked to the sequence(s) or gene(s) of interest, as well as a polyadenylation sequence.

Within certain embodiments of the invention, the nucleic acid expression vectors described herein may be contained within a plasmid construct. In addition to the components of the nucleic acid expression vector, the plasmid construct may also include a bacterial origin of replication, one or more selectable markers, a signal which allows the plasmid construct to exist as single-stranded DNA (e.g., a M13 origin of replication), a multiple cloning site, and a “mammalian” origin of replication (e.g., a SV40 or adenovirus origin of replication).

“Non-immunogenic” refers to a selectable marker or prodrug activating enzyme that does not cause an unwanted immune reaction in the majority of patients when it is administered as part of a gene delivery vehicle. Such genes may be human genes, non-human genes, or, mutated human genes that lack sufficient difference from normal human genes (normally less than 10% amino acid sequence difference), may be genes that although not of human origin do not carry epitopes that allow effective presentation of the protein sequence through MHC class I or class 2 presentation in patients, or may be genes that carry sequences that prevent the effective presentation of otherwise immunogenic epitopes. It is important to note that at least some non-immunogenic selectable markers will be species specific. In general, for clinical use, non-immunogenic markers will preferably be of human origin.

“Selectable marker” refers to genes that are included in a gene delivery vehicle and that have no therapeutic acitvity, but rather is included to allow for simpler preparation, manufacturing, characterization or testing of the gene delivery vehicle.

As noted above, the present invention provides compositions and methods for delivering a gene delivery vehicle to a warm-blooded animal. Within one aspect, such methods comprise the step of administering to a warm-blooded animal a gene delivery vehicle which directs the expression of a non-immunogenic selectable marker. Within other aspects, methods are provided for delivering a gene delivery vehicle to a warm-blooded animal, comprising the step of administering to a warm-blooded animal a gene delivery vehicle which directs the expression of a non-immunogenic molecule which is capable of activating an otherwise inactive compound into an active compound (i.e., a “prodrug activating enzyme” or “PAE”). As discussed in more detail below, a wide variety of non-immunogenic selectable markers/prodrug activating enzymes may be utilized within the context of the present invention.

A. Non-Immunogenic Markers/Prodrug Activating Enzymes

A wide variety of non-immunogenic markers and/or prodrug activating enzymes may be expressed by the gene delivery vehicles of the present invention. Briefly, the markers and PAE of the present invention may be readily tested for immunogenicity by a variety of assays, including for example, CTL assays for antigens to which the organism has previously generated immunity, and in vitro generation of T-cell response utilizing dendritic cells transduced with the antigen for antigens to which the organism does not have a previously existing response (see Henderson et al., Canc. Res. 56:3763-3770,1996; Hsu et al., Nat. Med. 2:52-58,1995; CTL assays can be conducted as described in WO 91/02805). Another method for ensuring that an antigen is non-immunogenic is to administer the antigen in a standard skin test such as one utilized to test allergic reactions. It should be noted however, that while the above tests may be utilized in order to ascertain markers or PAE which are non-immunogenic within the context of the present invention (i.e., do not produce statistically significant results), that some small percentage of patients may nevertheless react against the markers or PAE described herein.

Markers and PAEs of the present invention may be obtained from a variety of sources. For example, the marker or PAE may be, in its native state, a human enzyme, and thus, by its very nature be non-immunogenic. Similarly, markers or PAE closesly related species such as macaques may likewise be non-immunogenic. Within further embodiments of the invention, the marker or PAE may be of non-human origin, and can be made non-imunogenic by mutation (e.g., substition, deletion or insertion). Representative examples of such PAE's and associated prodrug molecules include Alkaline phosphatase and various toxic phosphorylated compounds such as phenolmustard phosphate, doxorubicin phosphate, mitomycin phosphate and etoposide phosphate; α-Galactosidase and N-[4-(α-D-galactopyranosyl) Benyloxycarbonyl]-daunorubicin; Azoreductase and azobenzene mustards; β-glucosidase and amygdalin; β-glucuronidase and phenolmustard-glucuronide and epirubicin-glucuronide; Carboxypeptidase A and methotrexate-alanine; Cytochrome P450 and cyclophosphamide or ifosfamide; DT diaphorase and 5-(Aziridine-1-yl)-2,4,dinitrobenzamide (CB1954) (Cobb et al., Biochem. Pharmacol 18:1519-1527, 1969; Knox et al., Cancer Metastasis Rev. 12:195-212, 1993; γ-glutamyl transferase and γ-glutamyl p-phenylenediamine mustard; Nitroreductase and CB1954 or derivatives of 4-Nitrobenzyloxycarbonyl; glucose oxidase and glucose; xanthine oxidase and hypoxanthine; and plasmin and peptidyl-p-phenylenediamine-mustard. Non-immunogenic markers or PAE's may also be made by expressing an enzyme in a compartment of the cell where it is not normally expressed. For example, the enzyme furin, normally expressed in the trans Golgi, can be made to express on the cell surface. There it can activate drugs than normally may not reach the trans-Golgi. In order to further a more complete understanding of such selectable markers and/or prodrugs, certain of these markers or prodrugs are discussed in more detail below.

1. Use of Human Deoxycytidine Kinase and Human Equilibrative Nucleoside Transporter as Novel Prodrugs for Tumor Therapy

Deoxycytidine kinase (dCK) is responsible for phosphorylation of several deoxynucleosides and their analogs. dCK has a broad substrate specificity for deoxycytidine, deoxyadenosine and deoxyguanosine and is important in the maintenance of normal dNTP pools. dCK also can phosphorylate a number of anti-tumor and anti-viral nucleoside analogs, including cytosine arabinoside (ara-C) and ddC. T-cells have relatively high levels of dCK activity, although in most other cell types the enzyme is found at low levels and is relatively unstable. The phosphorylation of deoxyadenosine and deoxyguanosine by dCK is the first step in the synthesis of dATP and dGTP which are utilized in DNA synthesis. The human deoxycytidine kinase mRNA contains an open reading frame of 780 nt and encodes a polypeptide with a predicted size of 30.5 kD. The cDNA was first cloned by Chottiner et al., PNAS 88:1531-1535, 1991.

2. Cytosine arabinoside (ara-C)

Ara-C is the prototype nucleoside chemotherapeutic drug and differs from its physiologic counterpart, deoxycytidine, by the presence of an additional-OH group at the 2′ position. Ara-C is the most effective agent in the treatment of acute myeloid leukemia. As a single agent, ara-C induces remission in 50% of patients with acute myeloblastic leukemia (AML). Ara-C is also used in blast crisis of chronic granulocytic leukemia (CGL), acute lymphocytic leukemia (ALL) and non-Hodgkins lymphoma. Ara-C incorporates into replicating DNA and terminates DNA strand elongation in dividing cells. Because of its selectivity for rapidly growing tumors and its propensity for deamination by cytosine deaminase, ara-C has not been effective for the treatment of solid tumors.

Ara-C enters cells via the equilbrative nucleoside transporter (hENT). Once in the cell, ara-C can undergo deamination to ara-U or serve as a substrate for salvage pathway enzymes to generate ara-CTP. Ara-CTP competes with dCTP and inhibits DNA polymerase. Intracellular metabolism of ara-C results in three sequential phosphorylation reactions. The first is mediated by dCK to form ara-CMP. dCMP kinase results in the formation of ara-CDP which is phosphorylated by nucleoside diphosphate kinase to generate ara-CTP. There are two limiting steps in the generation of ara-CTP from ara-C: the initial intracellular transport of ara-C by the membrane bound transporter (hENT) and intracellularly, the balance between deamidation by cytidine deaminase versus the initial phosphorylation event by deoxycytidine kinase. The intracellular generation of the toxic ara-CTP metabolite can be enhanced by either expression of the recently cloned hENT1 (Griffiths et al., Nature Medicine 3:89-93, 1997) transporter or increased expression of dCK. It is believed that dCK expression is the rate limiting step in ara-CTP formation intracellularly (Manome et al., Nature Medicine 2:567-573, 1996). The level of cell surface expression of hENTl imposes a rate limiting transport step on the accumulation of the toxic ara-CTP at drug concentrations that are used clinically (Wiley et al., J. Clin. Invest. 75:632-642, 1985). hENT1 is highly expressed in acute myeloid leukemia whereas normal leukocytes express low levels of hENT1. Co-expression of both of these molecules should have synergistic effects, especially in solid tumors where augmented tumor cell killing mediated by the so-called “bystander” effect will occur. Increased co-expression of hENT1 and dCK in tumor cells will allow therapeutic doses of ara-C to be reduced thereby reducing toxic side effects. Dose limiting toxicities include severe myelosuppression and gastrointestinal epithelial injury.

Because of the high levels of cytidine deaminase in the gastrointestinal epithelium and first pass elimination in the liver, ara-C is not given orally. However, when administered by IV infusion, the drug distributes rapidly in total body water and concentrations in the CSF reach 50% of that in plasma after 2 hours of continuous infusion. This latter feature of penetrating the blood brain barrier as well as relative lack of toxicity against post-mitotic cells makes ara-C an attractive candidate for the treatment of CNS tumors. Currently, ara-C is not widely used against solid tumors, however, potentiation of action of the drug will occur in cells that express augmented levels of dCK and hENT1. Plasma half life of ara-C is less than 20 minutes due to the rapid deamination reaction. Deamination is minimal in the CSF and ara-C is currently used intrathecally for treatment of meningeal leukemia.

3. Cyclophosphamide

Cyclophosphamide and its isomer ifosfamide are cell cycle-nonspecific alkylating agents that undergo bioactivation catalyzed by liver cytochrome P-450 enzymes. The therapeutic efficacy of these oxazaphosphorine anticancer drugs is limited by host toxicity resulting from the systemic distribution of activated drug metabolites formed in the liver (see, e.g., Chen and Waxman, Canc. Res. 55:581, 1994).

4. Cytochrome P-450

Biotransformation involves the metabolism of xenobiotics (pharmaceuticals, plant-derived chemicals, environmental pollutants, pesticides and herbicides) and occurs in the liver, where the xenobiotics are rendered inactive and water soluble prior to elimination. Two series of reactions occur: Phase I reactions result in the addition of a chemical group that can be further modified by the Phase II reaction involving hydrolysis or conjugation. The Phase I reactions are carried out by a group of enzymes called the cytochromes P-450 which are all endoplasmic reticulum integral membrane monooxygenases. The cytochrome P-450 enzymes interact with organic substrates (xenobiotics) resulting in the oxidation of the substrate and generation of water. NADPH is used as the electron donor and catalyzes the reaction. A cytochrome P-450 reductase catalyzes the reduction of the CYC P-450 monooxygenases. The CYC P-450 is a multigene superfamily whereas the reductase is the product of a single gene that interacts with all the CYC P-450s. The Phase II conjugation reactions are important in the detoxification of reactive compounds such as carcinogens. Normally, these reactive compounds are conjugated resulting in: glucuronidation; sulfation; methylation or glutathione conjugation or amino-acid conjugation.

In order to further describe certain preferred embodiments of the invention the cloning of an active human CYC P-450 gene into a retroviral vector is described below within the examples. Briefly, the subcellular localization of the CYC P-450 proteins is re-targeted to allow expression of the protein either extracellularly or bound to the inner surface of the plasma membrane. Xenobiotic compounds, including anti-cancer agents may undergo the Phase I oxidation reactions, however, they are not subjected to the Phase II detoxification conjugation reactions, thereby rendering the anti-cancer agents as active toxic metabolites. Because of the membrane permeability of these reactants, they may diffuse from cell to cell, resulting in a bystander effect.

B. Gene Delivery Vehicles

The non-immunogenic markers/PAE of the present invention may be utilized in a wide variety of gene delivery vehicles. As discussed in more detail below, the gene delivery vehicle may be of either viral or non-viral origin (See generally, Jolly, Cancer Gene Therapy 1 (1994) 51-64; Kimura, Human Gene Therapy 5 (1994) 845-852, Connelly, Human Gene Therapy 6 (1995) 185-193 and Kaplitt, Nature Genetics 6 (1994) 148-153).

1. Construction of Retroviral Gene Delivery Vehicles

Within one aspect of the present invention, retroviral vector constructs are provided which are constructed to carry or express a non-immunogenic selectable marker and/or PAE. Numerous retroviral gene delivery vehicles may be utilized within the context of the present invention, including for example those described in GB 2200651; EP 0415731; EP 0345242; WO 89/02468; WO 89/05349; WO 89/09271; WO 90/02806; WO 90/07936; WO 90/07936; WO 94/03622; WO 93/25698; WO 93/25234; WO 93/11230; WO 93/10218; WO 91/02805; in U.S. Pat. No. 5,219,740; U.S. Pat. No. 4,405,712; U.S. Pat. No. 4,861,719; U.S. Pat. No. 4,980,289 and U.S. Pat. No. 4,777,127; in U.S. Ser. No. 07/800,921 and provisional application 60/053066, filed Jul. 18, 1997; and in Vile, Cancer Res 53:3860-3864, 1993; Vile, Cancer Res 53:962-967, 1993; Ram, Cancer Res 53:83-88, 1993; Takamiya, J Neurosci Res 33:493-503, 1992; Baba, J Neurosurg 79:729-735, 1993; Mann, Cell 33:153, 1983; Cane, Proc Natl Acad Sci 81;6349, 1984; and Miller, Human Gene Therapy 1, 1990.

Retroviral gene delivery vehicles of the present invention may be readily constructed from a wide variety of retroviruses, including for example, B, C, and D type retroviruses as well as spumaviruses and lentiviruses (see RNA Tumor Viruses, Second Edition, Cold Spring Harbor Laboratory, 1985). Briefly, viruses are often classified according to their morphology as seen under electron microscopy. Type “B” retroviruses appear to have an eccentric core, while type “C” retroviruses have a central core. Type “D” retroviruses have a morphology intermediate between type B and type C retroviruses. Representative examples of suitable retroviruses include those described in RNA Tumor Viruses: Molecular Biology of tumor viruses, Second Edition, Cold Spring Harbor Laboratory, 1985 at pages 2-7, as well as a variety of xenotropic retroviruses (e.g., NZB-X1, NZB-X2 and NZB₉₋₁ (see O'Neill et al., J Vir. 53:100-106, 1985)) and polytropic retroviruses (e.g., MCF and MCF-MLV (see Kelly et al., J Vir. 45(1): 291-298, 1983)). Such retroviruses may be readily obtained from depositories or collections such as the American Type Culture Collection (“ATCC”; Rockville, Md.), or isolated from known sources using commonly available techniques.

Particularly preferred retroviruses for the preparation or construction of retroviral gene delivery vehicles of the present invention include retroviruses selected from the group consisting of Avian Leukosis Virus, Bovine Leukemia Virus, Murine Leukemia Virus, Mink-Cell Focus-Inducing Virus, Murine Sarcoma Virus, Gibbon Ape Leukemia Virus, Feline Leukemia Virus, Reticuloendotheliosis virus and Rous Sarcoma Virus. Particularly preferred Murine Leukemia Viruses include 4070A and 1504A (Hartley and Rowe, J Virol. 19:19-25, 1976), Abelson (ATCC No. VR-999), Friend (ATCC No. VR-245), Graffi, Gross (ATCC No. VR-590), Kirsten, Harvey Sarcoma Virus and Rauscher (ATCC No. VR-998), and Moloney Murine Leukemia Virus (ATCC No. VR-190). Particularly preferred Rous Sarcoma Viruses include Bratislava, Bryan high titer (e.g., ATCC Nos. VR-334, VR-657, VR-726, VR-659, and VR-728), Bryan standard, Carr-Zilber, Engelbreth-Holm, Harris, Prague (e.g., ATCC Nos. VR-772, and 45033), HIV, HIV-1, HIV-2, SIV, FIV, and Schmidt-Ruppin (e.g., ATCC Nos. VR-724, VR-725, VR-354).

Any of the above retroviruses may be readily utilized in order to assemble or construct retroviral gene delivery vehicles given the disclosure provided herein, and standard recombinant techniques (e.g., Sambrook et al., Molecular Cloning: A Laboratory Manual, 2d ed., Cold Spring Harbor Laboratory Press, 1989; Kunkle, PNAS 82:488, 1985). In addition, within certain embodiments of the invention, portions of the retroviral gene delivery vehicles may be derived from different retroviruses. For example, within one embodiment of the invention, retroviral vector LTRs may be derived from a Murine Sarcoma Virus, a tRNA binding site from a Rous Sarcoma Virus, a packaging signal from a Murine Leukemia Virus, and an origin of second strand synthesis from an Avian Leukosis Virus.

Within one aspect of the present invention, retroviral vector constructs are provided comprising a 5′ LTR, a tRNA binding site, a packaging signal, one or more heterologous sequences, an origin of second strand DNA synthesis and a 3′ LTR, wherein the vector construct lacks gag/pol or env coding sequences. Representative examples of such vector constructs are described within PCT application Nos. US 95/05789 and US 97/07697.

Packaging cell lines suitable for use with the above described retroviral vector constructs may be readily prepared (see U.S. application Ser. No. 08/240,030 and U.S. application Ser. No. 07/800,921; as well as PCT application Nos. US 95/05789 and US 97/07697), and utilized to create producer cell lines (also termed vector cell lines or “VCLs”) for the production of recombinant vector particles. Within preferred embodiments, transduced packaging cell lines can be selected utilizing a number of titering methods, including PCR titering (see, e.g., Example 5A), or by staining of transduced cells for an appropriate transferred marker (e.g., Fast Red staining as described in Example 5B).

2. Alphavirus gene delivery vehicles

The present invention also provides a variety of alphavirus-based vectors which can function as gene delivery vehicles. Such vectors can be constructed from a wide variety of alphaviruses, including for example, Sindbis viruses vectors, Semliki Forest virus (ATCC VR-67; ATCC VR-1247), Ross River virus (ATCC VR-373; ATCC VR-1246) and Venezuelan equine encephalitis virus (ATCC VR923; ATCC VR-1250; ATCC VR-1249; ATCC VR-532).

As an example, the Sindbis virus, which is the prototype member of the alphavirus genus of the togavirus family is an unsegmented genomic RNA (49S RNA) of virus of approximately 11,703 nucleotides in length. This virus contains a 5′ cap and a 3′ poly-adenylated tail, and displays positive polarity. Infectious enveloped Sindbis virus is produced by assembly of the viral nucleocapsid proteins onto the viral genomic RNA in the cytoplasm and budding through the cell membrane embedded with viral encoded glycoproteins. Entry of virus into cells is by endocytosis through clatharin coated pits, fusion of the viral membrane with the endosome, release of the nucleocapsid, and uncoating of the viral genome. During viral replication the genomic 49S RNA serves as template for synthesis of the complementary negative strand. This negative strand in turn serves as template for genomic RNA and an internally initiated 26S subgenomic RNA. The Sindbis viral nonstructural proteins are translated from the genomic RNA while structural proteins are translated from the subgenomic 26S RNA. All viral genes are expressed as a polyprotein and processed into individual proteins by post translational proteolytic cleavage. The packaging sequence resides within the nonstructural coding region, therefore only the genomic 49S RNA is packaged into virions.

A variety of different alphavirus vector systems may be constructed and utilized within the present invention. Representative examples of such systems include those described in U.S. patent application Ser. Nos. 08/198,450, 08/405,627 and 08/679,640, U.S. Pat. Nos 5,091,309; 5,217,879 and 5,185440, PCT patent application publication numbers WO 92/10578, WO/94/21792, WO 95/27069, WO 95/27044 and WO 95/07994, and PCT application No. US 97/06010.

Particularly preferred alphavirus vectors for use within the present invention include those which are described within U.S. application Ser. No. 08/198,450. Briefly, within one embodiment, alphavirus vector constructs are provided comprising a 5′ sequence which is capable of initiating in vitro transcription of a alphavirus RNA, a nucleotide sequence encoding alphavirus non-structural proteins, a viral junction region which is active, modified to reduce viral transcription of the subgenomic fragment, or inactivated such that viral transcription of the subgenomic fragment is prevented, and a alphavirus RNA polymerase recognition sequence.

In still further embodiments, the vector constructs described above contain no alphavirus structural proteins in the vector constructs. The selected heterologous sequence may be located downstream from the viral junction region; in the vector constructs having a second viral junction, the selected heterologous sequence may be located downstream from the second viral junction region, where the heterologous sequence is located downstream, the vector construct may comprise a polylinker located between the viral junction region and said heterologous sequence, and preferably the polylinker does not contain a wild-type Sindbis virus restriction endonuclease recognition sequence.

3. Other Viral Gene Delivery Vehicles

In addition to retroviral vectors and alphavirus-based vectors, numerous other viral vectors systems may also be utilized as a gene delivery vehicle. For example, within one embodiment of the invention adenoviral vectors may be employed as a gene delivery vehicle. Representative examples of such vectors include those described by, for example, Berkner, Biotechniques 6:616-627, 1988; Rosenfeld et al., Science 252:431-434, 1991; WO 93/9191; Kolls et al., PNAS 91(1): 215-219, 1994; Kass-Eisler et al., PNAS 90(24): 11498-502, 1993; Guzman et al., Circulation 88(6): 2838-48, 1993; Guzrnan et al., Cir. Res. 73(6): 1202-1207, 1993; Zabner et al., Cell 75(2): 207-216, 1993; Li et al., Hum. Gene Ther. 4(4): 403-409, 1993; Caillaud et al., Eur. J Neurosci. 5(10): 1287-1291, 1993; Vincent et al., Nat. Genet. 5(2): 130-134, 1993; Jaffe et al., Nat. Genet. 1(5): 372-378, 1992; and Levrero et al., Gene 101(2): 195-202, 1991; and WO 93/07283; WO 93/06223; and WO 93/07282. Exemplary known adenoviral gene therapy vectors employable in this invention include those described in the above referenced documents and in WO 94/12649, WO 93/03769, WO 93/19191, WO 94/28938, WO 95/11984, WO 95/00655, WO 95/27071, WO 95/29993, WO 95/34671, WO 96/05320, WO 94/08026, WO 94/11506, WO 93/06223, WO 94/24299, WO 95/14102, WO 95/24297, WO 95/02697, WO 94/28152, WO 94/24299, WO 95/09241, WO 95/25807, WO 95/05835, WO 94/18922 and WO 95/09654. Alternatively, administration of DNA linked to killed adenovirus as described in Curiel, Hum. Gene Ther. 3:147-154, 1992 may be employed.

Gene delivery vehicles of the present invention also include parvovirus such as adenovirus associated virus (AAV) vectors. Representative examples of such vectors include the AAV vectors disclosed by Srivastava in WO 93/09239, Samulski et al., J. Vir. 63:3822-3828, 1989; Mendelson et al., Virol. 166:154-165, 1988; Flotte et al., PNAS 90(22): 10613-10617, 1993. Particularly preferred AAV vectors comprise the two AAV inverted terminal repeats in which the native D-sequences are modified by substitution of nucleotides, such that at least 5 native nucleotides and up to 18 native nucleotides, preferably at least 10 native nucleotides up to 18 native nucleotides, most preferably 10 native nucleotides are retained and the remaining nucleotides of the D-sequence are deleted or replaced with non-native nucleotides. The native D-sequences of the AAV inverted terminal repeats are sequences of 20 consecutive nucleotides in each AAV inverted terminal repeat (i.e., there is one sequence at each end) which are not involved in HP formation. The non-native replacement nucleotide may be any nucleotide other than the nucleotide found in the native D-sequence in the same position. Other employable exemplary AAV vectors are pWP-19, pWN-1, both of which are disclosed in Nahreini, Gene 124:257-262, 1993. Another example of such an AAV vector is psub20l. See Samulski, J. Virol. 61:3096, 1987. Another exemplary AAV vector is the Double-D ITR vector. How to make the Double D ITR vector is disclosed in U.S. Pat. No. 5,478,745. Still other vectors are those disclosed in Carter, U.S. Pat. No. 4,797,368 and Muzyczka, U.S. Pat. No. 5,139,941; Chartejee, U.S. Pat. No. 5,474,935; and Kotin, PCT Patent Publication WO 94/288157. Yet a further example of an AAV vector employable in this invention is SSV9AFABTKneo, which contains the AFP enhancer and albumin promoter and directs expression predonimantly in the liver. Its structure and how to make it are disclosed in Su, Human Gene Therapy 7:463-470, 1996. Additional AAV gene therapy vectors are described in U.S. Pat. Nos. 5,354,678; 5,173,414; 5,139,941; and 5,252,479.

Gene delivery vehicles of the present invention also include herpes vectors. Representative examples of such vectors include those disclosed by Kit in Adv. Exp. Med. Biol. 215:219-236, 1989; and those disclosed in U.S. Pat. No. 5,288,641 and EP 0176170 (Roizman). Additional exemplary herpes simplex virus vectors include HFEM/ICP6-LacZ disclosed in WO 95/04139 (Wistar Institute), pHSVlac described in Geller, Science 241:1667-1669, 1988 and in WO 90/09441 and WO 92/07945; HSV Us3:: pgC-lacZ described in Fink, Human Gene Therapy 3:11-19, 1992; and HSV 7134, 2 RH 105 and GAL4 described in EP 0453242 (Breakefield), and those deposited with the ATCC as accession numbers ATCC VR-977 and ATCC VR-260.

Gene delivery vehicles may also be generated from a wide variety of other viruses, including for example, poliovirus (Evans et al., Nature 339:385-388, 1989; and Sabin, J. Biol. Standardization 1:115-118, 1973); rhinovirus; pox viruses, such as canary pox virus or vaccinia virus (Fisher-Hoch et al., PNAS 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330 and 5,017,487; WO 89/01973); SV40 (Mulligan et al., Nature 277:108-114, 1979); influenza virus (Luytjes et al., Cell 59:1107-1113, 1989; McMicheal etal., N. Eng. J Med. 309:13-17, 1983; and Yap et al., Nature 273:238-239, 1978); SV40; HIV (Poznansky, J Virol. 65:532-536, 1991); measles (EP 0 440,219); astrovirus (Munroe, S. S. et al., J. Vir. 67:3611-3614, 1993); and coronavirus, as well as other viral systems (e.g, EP 0,440,219; WO 92/06693; U.S. Pat. No. 5,166,057). In addition, viral carriers may be homologous, non-pathogenic(defective), replication competent virus (e.g., Overbaugh et al., Science 239:906-910, 1988), and nevertheless induce cellular immune responses, including CTL.

4. Non-viral Gene Delivery Vehicles

In addition to the above viral-based vectors, numerous non-viral gene delivery vehicles may likewise be utilized within the context of the present invention. For example, within one embodiment of the invention the gene delivery vehicles is a eukarytic layered expression systems (see WO 95/07994 for a detailed description of eukaryotic layered expression systems).

Other gene delivery vehicles and methods that may be employed such as, for example, nucleic acid expression vectors; polycationic condensed DNA linked or unlinked to killed adenovirus alone, for example see U.S. Ser. No. 08/366,787, filed Dec. 30, 1994 and Curiel, Hum Gene Ther 3:147-154, 1992; ligand linked DNA, for example see Wu, J. Biol Chem 264:16985-16987, 1989; eucaryotic cell delivery vehicles cells, for example see U.S. Ser. No.08/240,030, filed May 9, 1994, and U.S. Ser. No. 08/404,796; deposition of photopolymerized hydrogel materials; hand-held gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip, Mol Cell Biol 14:2411-2418, 1994 and in Woffendin, Proc Natl Acad Sci 91:1581-1585, 1994.

Particle mediated gene transfer may be employed, for example see U.S. Ser. No. 60/023,867. Briefly, the sequence of interest can be inserted into conventional vectors that contain conventional control sequences for high level expression, and then be incubated with synthetic gene transfer molecules such as polymeric DNA-binding cations like polylysine, protamine, and albumin, linked to cell targeting ligands such as asialoorosomucoid, as described in Wu and Wu, J. Biol. Chem. 262:4429-4432, 1987, insulin as described in Hucked, Biochem Pharmacol 40:253-263, 1990, galactose as described in Plank, Bioconjugate Chem 3:533-539, 1992 lactose or transferrin.

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm.

Liposomes that can act as gene delivery vehicles are described in U.S. Pat. No. 5,422,120, WO 95/13796, WO 94/23697, WO 91/144445 and EP 524,968. As described in U.S. Ser. No. 60/023,867, nucleic acid sequences can be inserted into conventional vectors that contain conventional control sequences for high level expression, and then be incubated with synthetic gene transfer molecules such as polymeric DNA-binding cations like polylysine, protamine, and albumin, linked to cell targeting ligands such as asialoorosomucoid, insulin, galactose, lactose, or transferrin. Other delivery systems include the use of liposomes to encapsulate DNA comprising the gene under the control of a variety of tissue-specific or ubiquitously-active promoters. Further non-viral delivery suitable for use includes mechanical delivery systems such as the approach described in Woffendin et al., Proc. Natl. Acad. Sci. USA 91(24): 11581-11585, 1994. Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and WO 92/11033 Exemplary liposome and polycationic gene delivery vehicles are those described in U.S. Pat. Nos. 5,422,120 and 4,762,915, in WO 95/13796, WO 94/23697, and WO 91/14445, in EP 0524968 and in Starrier, Biochemistry, pages 236-240 (1975) W. H. Freeman, San Francisco; Shokai, Biochem Biophys Acct 600:1, 1980; Bayer, Biochem Biophys Acct 550:464, 1979; Rivet, Meth Enzyme 149:119, 1987; Wang, Proc Natl Acad Sci84:7851, 1987; Plant, Anal Biochem 176:420, 1989.

D. Adept

Within other aspects of the present invention, the prodrugs described herein may be linked to an antibody, and utilized for antibody-directed enzyme prodrug therapy essentially as described in WO 95/13095.

E. Heterologous Sequences

Any of the gene delivery vehicles described above may include, contain (and/or express) one or more heterologous sequences, as well as the non-immunogenic selectable marker or PAE. A wide variety of heterologous sequences may be utilized within the context of the present invention, including for example, cytotoxic genes, disease-associated antigens, antisense sequences, sequences which encode gene products corresponding to genetic deficiencies that need to be expressed over a long period of time (greater than 2-4 weeks), sequences which encode immunogenic portions of disease-associated antigens and sequences which encode immune accessory molecules. Representative examples of cytotoxic genes include the genes which encode proteins such as ricin (Lamb et al., Eur. J Biochem. 148:265-270, 1985), diphtheria toxin (Tweten et al., J Biol. Chem. 260:10392-10394, 1985), cholera toxin (Mekalanos et al., Nature 306:551-557, 1983; Sanchez & Holmgren, PNAS 86:481-485, 1989), and Pseudomonas exotoxin A (Carroll and Collier, J. Biol. Chem. 262:8707-8711, 1987).

Within further embodiments of the invention, antisense RNA may be utilized as a cytotoxic gene in order to induce a potent Class I restricted response. Briefly, in addition to binding RNA and thereby preventing translation of a specific mRNA, high levels of specific antisense sequences may be utilized to induce the increased expression of interferons (including gamma-interferon), due to the formation of large quantities of double-stranded RNA. The increased expression of gamma interferon, in turn, boosts the expression of MHC Class I antigens. Preferred antisense sequences for use in this regard include actin RNA, myosin RNA, and histone RNA. Antisense RNA which forms a mismatch with actin RNA is particularly preferred.

Within further aspects of the present invention, gene delivery vehicles of the present invention may also direct the expression of one or more sequences which encode immunogenic portions of disease-associated antigens. As utilized within the context of the present invention, antigens are deemed to be “disease-associated” if they are either associated with rendering a cell (or organism) diseased, or are associated with the disease-state in general but are not required or essential for rendering the cell diseased. In addition, antigens are considered to be “immunogenic” if they are capable, under appropriate conditions, of causing an immune response (either cell-mediated or humoral). Immunogenic “portions” may be of variable size, but are preferably at least 9 amino acids long, and may include the entire antigen.

A wide variety of “disease-associated” antigens are contemplated within the scope of the present invention, including for example immunogenic, non-tumorigenic forms of altered cellular components which are normally associated with tumor cells (see U.S. application Ser. No. 08/104,424). Representative examples of altered cellular components which are normally associated with tumor cells include ras* (wherein “*” is understood to refer to antigens which have been altered to be non-tumorigenic), p53*, and Rb*.

“Disease-associated” antigens should also be understood to include all or portions of various eukaryotic (including for example, parasites), prokaryotic (e.g., bacterial) or viral pathogens. Representative examples of viral pathogens include the Hepatitis B Virus (“HBV”) and Hepatitis C Virus (“HCV;” see U.S. application Ser. No. 08/102/132), Human Papiloma Virus (“HPV;” see WO 92/05248; WO 90/10459; EPO 133,123), Epstein-Barr Virus (“EBV;” see EPO 173,254; JP 1,128,788; and U.S. Pat. Nos. 4,939,088 and 5,173,414), Feline Leukemia Virus (“FeLV;” see U.S. application Ser. No. 07/948,358; EPO 377,842; WO 90/08832; WO 93/09238), Feline Immunodeficiency Virus (“FIV;” U.S. Pat. No. 5,037,753; WO 92/15684; WO 90/13573; and JP 4,126,085), HTLV I and II, and Human Immunodeficiency Virus (“HIV;” see U.S. application Ser. No. 07/965,084).

Within other aspects of the present invention, the gene delivery vehicles described above may also direct the expression of one or more immune accessory molecules. As utilized herein, the phrase “immune accessory molecules” refers to molecules which can either increase or decrease the recognition, presentation or activation of an immune response (either cell-mediated or humoral). Representative examples of immune accessory molecules include IL-1, IL-2, IL-3, IL-4, IL-5, IL-6, IL-7 (U.S. Pat. No. 4,965,195), IL-8, IL-9, IL-10, IL-11, IL-12 (Wolf et al., J. Immun. 46:3074, 1991; Gubler et al., PNAS 88:4143, 1991; WO 90/05147; EPO 433,827), IL-13 (WO 94/04680), IL-15 or ETF, GM-CSF, M-CSF-1, G-CSF, CD3 (Krissanen et al., Immunogenetics 26:258-266, 1987), CD8, ICAM-1 (Simmons et al., Nature 331:624-627, 1988), ICAM-2 (Singer, Science 255:1671, 1992), β-microglobulin (Parnes et al., PNAS 78:2253-2 al., Nature 338:521, 1989), LFA3 (Wallner et al., J Exp. Med. 166(4): 923-932, 1987), HLA Class I, HLA Class II molecules B7 (Freeman et al., J. Immun. 143:2714, 1989), and B7-2. Within a preferred embodiment, the heterologous gene encodes gamma-interferon.

Within preferred aspects of the present invention, the gene delivery vehicles described herein may direct the expression of more than one heterologous sequence. Such multiple sequences may be controlled either by a single promoter, or preferably, by additional secondary promoters (e.g., Internal Ribosome Binding Sites or “IRBS”). Within preferred embodiments of the invention, a gene delivery vehicle directs the expression of heterologous sequences which act synergistically. For example, within one embodiment retroviral vector constructs are provided which direct the expression of a molecule such as IL-12, IL-2, gamma interferon, or other molecule which acts to increase cell-mediated presentation in the T_{H}1 pathway, along with an immunogenic portion of a disease-associated antigen. In such embodiments, immune presentation and processing of the disease-associated antigen will be increased due to the presence of the immune accessory molecule.

Within other aspects of the invention, gene delivery vehicles are provided which direct the expression of one or more heterologous sequences which encode “replacement” genes. As utilized herein, it should be understood that the term “replacement genes” refers to a nucleic acid molecule which encodes a therapeutic protein that is capable of preventing, inhibiting, stabilizing or reversing an inherited or noninherited genetic defect. Representative examples of such genetic defects include disorders in metabolism, immune regulation, hormonal regulation, and enzymatic or membrane associated structural function. Representative examples of diseases caused by such defects include Cystic Fibrosis (due to a defect in the Cystic Fibrosis Transmembrane Conductance Regulator (“CFTCR”), see Dorin et al., Nature 326:614, Parkinson's Disease, Adenosine Deaminase deficiency (“ADA;” Hahma et al., J. Bact. 173:3663-3672, 1991), β-globin disorders, Hemophilia A & B (Factor VIII-deficiencies, see Wood et al., Nature 312:330, 1984), Factor IX deficiencies, Gaucher disease, diabetes, forms of gouty arthritis and Lesch-Nylan disease (due to “HPRT” deficiencies; see Jolly et al., PNAS 80:477-481, 1983) Duchennes Muscular Dystrophy and Familial Hypercholesterolemia (LDL Receptor mutations; see Yamamoto et al., Cell 39:27-38, 1984) and Gaucher's Syndrome.

Sequences which encode the above-described heterologous genes may be readily obtained from a variety of sources. For example, plasmids which contain sequences that encode immune accessory molecules may be obtained from a depository such as the American Type Culture Collection (ATCC, Rockville, Md.), or from commercial sources such as British Bio-Technology Limited (Cowley, Oxford England). Representative sources sequences which encode the above-noted immune accessory molecules include BBG 12 (containing the GM-CSF gene coding for the mature protein of 127 amino acids), BBG 6 (which contains sequences encoding gamma interferon), ATCC No. 39656 (which contains sequences encoding TNF), ATCC No. 20663 (which contains sequences encoding alpha interferon), ATCC Nos. 31902, 31902 and 39517 (which contains sequences encoding beta interferon), ATCC No 67024 (which contains a sequence which encodes Interleukin-1), ATCC Nos. 39405, 39452, 39516, 39626 and 39673 (which contains sequences encoding Interleukin-2), ATCC Nos. 59399, 59398, and 67326 (which contain sequences encoding Interleukin-3), ATCC No. 57592 (which contains sequences encoding Interleukin-4), ATCC Nos. 59394 and 59395 (which contain sequences encoding Interleukin-5), and ATCC No. 67153 (which contains sequences encoding Interleukin-6). It will be evident to one of skill in the art that one may utilize either the entire sequence of the protein, or an appropriate portion thereof which encodes the biologically active portion of the protein.

Alternatively, known cDNA sequences which encode cytotoxic genes or other heterologous genes may be obtained from cells which express or contain such sequences. Briefly, within one embodiment mRNA from a cell which expresses the gene of interest is reverse transcribed with reverse transcriptase using oligo dT or random primers. The single stranded cDNA may then be amplified by PCR (see U.S. Pat. Nos. 4,683,202, 4,683,195 and 4,800,159. See also PCR Technology: Principles and Applications for DNA Amplification, Erlich (ed.), Stockton Press, 1989 all of which are incorporated by reference herein in their entirety) utilizing oligonucleotide primers complementary to sequences on either side of desired sequences. In particular, a double stranded DNA is denatured by heating in the presence of heat stable Taq polymerase, sequence specific DNA primers, ATP, CTP, GTP and TTP. Double-stranded DNA is produced when synthesis is complete. This cycle may be repeated many times, resulting in a factorial amplification of the desired DNA.

Sequences which encode the above-described genes may also be synthesized, for example, on an Applied Biosystems Inc. DNA synthesizer (e.g., ABI DNA synthesizer model 392 (Foster City, Calif.)).

F. Compositions

Within other aspects of the present invention, any of the above gene delivery vehicles are provided in combination with a pharmaceutically acceptable carrier or diluent. Such pharmaceutical compositions may be prepared either as a liquid solution, or as a solid form (e.g., lyophilized) which is suspended in a solution prior to administration. In addition, the composition may be prepared with suitable carriers or diluents for topical administration, injection, or nasal, oral, vaginal, sub-lingual, inhalant or rectal administration.

Pharmaceutically acceptable carriers or diluents are nontoxic to recipients at the dosages and concentrations employed. Representative examples of carriers or diluents for injectable solutions include water, isotonic saline solutions which are preferably buffered at a physiological pH (such as phosphate-buffered saline or Tris-buffered saline), mannitol, dextrose, glycerol, and ethanol, as well as polypeptides or proteins such as human serum albumin. A particularly preferred composition comprises a retroviral vector construct or recombinant viral particle in 10 mg/ml mannitol, 1 mg/ml HSA, 20 mM Tris, pH 7.2, and 150 mM NaCl. In this case, since the recombinant vector represents approximately 1 mg of material, it may be less than 1% of high molecular weight material, and less than 1/100,000 of the total material (including water). This composition is stable at −70 C. for at least six months.

Pharmaceutical compositions of the present invention may also additionally include factors which stimulate cell division, and hence, uptake and incorporation of a gene delivery vehicle. Representative examples include Melanocyte Stimulating Hormone (MSH), for melanomas or epidermal growth factor for breast or other epithelial carcinomas. In addition, pharmaceutical compositions of the present invention may be placed within containers or kits (e.g., one container for the coupled targeting element, and a second container for the coupled gene delivery vehicle), along with packaging material which provides instructions regarding the use of such pharmaceutical compositions. Generally, such instructions will include a tangible expression describing the reagent concentration, as well as within certain embodiments, relative amounts of excipient ingredients or diluents (e.g., water, saline or PBS) which may be necessary to reconstitute the pharmaceutical compositions.

Particularly preferred methods and compositions for preserving certain of the gene delivery vehicles provided herein, such as recombinant viruses, are described in U.S. applications entitled “Methods for Preserving Recombinant Viruses” (U.S. application Ser. No. 08/135,938, filed Oct. 12, 1993, and U.S. application Ser. No. 08/152,342, filed Nov. 15, 1993, which are incorporated herein by reference in their entirety).

G. Methods of Treatment/Administration

As noted above, the present invention provides compositions and methods for delivering a gene delivery vehicles to a warm-blooded animal. Within one aspect, such methods comprise the step of administering to a warm-blooded animal a gene delivery vehicle which directs the expression of a non-immunogenic selectable marker. Within other aspects, methods are provided for delivering a gene delivery vehicle to a warm-blooded animal, comprising the step of administering to a warm-blooded animal a gene delivery vehicle which directs the expression of a non-immunogenic molecule which is capable of activating an otherwise inactive compound into an active compound. As utilized herein, it should be understood that “administering” refers not only to direct adminstration of a gene delivery vehicle (e.g., by direct injection or intravenous administration), but also to ex vivo routes wherein cells are removed from a donor, transduced or transfected with a gene delivery vehicle, and then introduced into the warm-blooded animal.

As discussed in more detail below, such methods may be utilized not only for delivering a desired heterologous sequence to cells, but for ablative therapy, as a fail-safe to lessen the risk of gene therapy, or for the transduction of cells which have been isolated from the body (e.g., T cells, cancer cells, or, stem cells).

1. Ablative Therapy

Prodrug activating genes can be used to ablate cancerous or hyperproliferative tissue such as in benign prostate hyperplasia, arthritic joints, smooth muscle proliferation in restinosis or immune cell proliferation in autoimmune disease. In any case where expression of the ablative gene is necessary for more than a few days (3-10), or it may be necessary to readminister the ablative gene it will be advantageous to use prodrug activating genes that do not elicit an immune response, in this case human genes or genes closely related to human genes (<10% difference in sequence).

2. Fail-safe Utility of Prodrug Activating Genes

Gene therapy has been proposed for many disease therapies including cancer, infectious diseases, autoimmune disease including graft versus host disease, cardiovascular disease, connective tissue disease, neurological disease, genetic disease and others. In all cases there is a least some risk involved in adding genes temporarily or permanently to the cells in a patient's body. One way to lessen that risk is to add a gene that is not itself toxic but the product of which can metabolize a prodrug into an active form that kills or inhibits the undesirable function or proliferation of the transduced cells. This approach can also be used to simply control cellular proliferation etc. of cells that have been manipulated (without gene therapy) and have the potential to be abnormal or cause pathology. However, if the period of activity of the transduced cells is larger than a few days or if repeat treatments are needed, the prodrug activating enzyme can cause an unwanted immune response that will destroy the cells that express them. This is not desirable in most cases. Therefore, the use of genes for the prodrug activating enzyme from human sources or from alternative sources that are very similar (<10% different in amino acid sequence) will allow the timing of cell ablation to be controlled by the physician, not by the immune system.

3. T Cell Transduction Methodology

T cell Transduction Allogeneic donor T cells are routinely used in allogeneic bone marrow (hemopoietic stem cells, HSC) transplants, mainly for lymphomas and leukemias. These T cells donate a immediate immune capability and cause increased cytokine production that aids engraftment, but they can also lead to graft versus host disease (GVHD), that currently kills about 1/3 of patients.

Retroviral vectors encoding prodrug activating enzymes are prepared as described in “Production and administration of High titer recombinant retroviruses” U.S. application Ser. No. 08/367,671, or by other means known to those skilled in the art. T cells can be transduced as described in “High efficiency ex vivo transduction of cells by high titer recombinant retroviral preparations (U.S. application Ser. No. 08/425,180). Other methods of growing and transducing T cells can be used and are known to those skilled in the art (e.g., A. S. Chuck and B. O. Palsson, Hum. Gene Ther. 7:743, 1996; Heslop et al., Nature Med 2:551, 1996; S. R. Riddell et al., Nature Medicine 2:216, 1996). T cells can also be transduced by methods used to grow and transduce T cells from HIV patients (e.g., T. Vandenddriessche et al., J. Virol. 69:4045, 1995; L. Q. Sun et al., PNAS 92:7272, 1995).

Within various embodiments of the invention, the above-described gene delivery vehicles or pharmaceutical compositions may be administered in vivo, or ex vivo. Representative routes for in vivo administration include intradermally (“i.d.”), intracranially (“i.c.”), intraperitoneally (“i.p.”), intrathecally (“i.t.”), intravenously (“i.v.”), subcutaneously (“s.c.”), intramuscularly (“i.m.”) or even directly into a tumor or the peri-tumoral area.

The above-described methods for sequential administration may be readily utilized for a variety of therapeutic (and prophylactic) treatments. For example, within one embodiment of the invention, the methods described above may be accomplished in order to inhibit or destroy a pathogenic agent in a warm-blooded animal. Such pathogenic agents include not only foreign organisms such as parasites, bacteria, and viruses, but cells which are “foreign” to the host, such as cancer or tumor cells, or other cells which have been “altered.” Within a preferred embodiment of the invention, the compositions described above may be utilized in order to directly treat pathogenic agents such as a tumor, for example, by direct injection into several different locations within the body of tumor. Alternatively, arteries which serve a tumor may be identified, and the compositions injected into such an artery, in order to deliver the compositions directly into the tumor. Within another embodiment, a tumor which has a necrotic center may be aspirated, and the compositions injected directly into the now empty center of the tumor. Within yet another embodiment, the above-described compositions may be directly administered to the surface of the tumor, for example, by application of a topical pharmaceutical composition containing the retroviral vector construct, or preferably, a recombinant retroviral particle.

Within other aspects of the present invention, methods are provided for generating an immune response against an immunogenic portion of an antigen, in order to prevent or treat a disease (see, e.g., U.S. application Ser. Nos. 08/104,424; 08/102,132, 07/948,358; 07/965,084), for suppressing graft rejection, (see U.S. application Ser. No. 08/116,827), for suppressing an immune response (see U.S. application Ser. No. 08/116,828), and for suppressing an autoimmune response (see U.S. application Ser. No. 08/116,983), utilizing the above-described compositions.

In addition, although warm-blooded animals (e.g., mammals or vertebrates such as humans, macaques, horses, cows, swine, sheep, dogs, cats, chickens, rats and mice) have been exemplified in the methods described above, such methods are also readily applicable to a variety of other animals, including, for example, fish.

4. Long Term Expression

Within certain embodiments of the invention, the gene delivery vehicles provided herein are administered in order to generate a sustained, long-term systemic expresssion of therapeutic genes expressed by the gene delivery vehicle. Preferably, long-term in vivo systemic expression is obtained by intravenous delivery methods or other in vivo or ex vivo methods as is described in detail below. For long term expression from a retroviral vector in vivo, the action of human complement on the retroviral vector is suppressed. This can be done by a variety of techniques known to one of skill in the art. Preferably, human packaging cell lines are used in order to inhibit the action of human complement on the retroviral vector particles (see PCT publication number US 91/06852, entitled “Packaging Cells”).

The terms “long term systemic expression” or “sustained systemic expression” as used herein in reference to in vivo expression of protein encoded by a gene delivery vehicle mean measurable or biologically active expression for 30 days, more preferably for 60 days, yet more preferably for 90 days, and still more preferably for six months after administration of the retroviral vector particle to a host. The term “measurable expression” as used herein in reference to in vivo expression of a protein encoded by a retroviral vector means that the protein is produced in sufficient amounts so as to be detectable in biological fluids such as serum or in cells such as stem cells, T-cells, and the like, by an assay specific for the expressed protein. The term “systemic expression” as used herein means that the proteins are expressed into the circulation and are thus useful for treatment of certain diseases. A variety of diseases discussed in detail below are amenable to treatment by this type of gene therapy.

For example, measurement of human growth hormone can be determined by an ELISA assay specific for human growth hormone protein. The term “biologically active expression” or “protein expression in biologically or therapeutically active amounts” when used herein in reference to in vivo expression of a protein encoded by a gene delivery vehicle vector means that protein is produced in sufficient amounts so as to be detectable in a functional assay. For example, expression of factor VIII can be measured in a clotting assay. Similarly other expressed proteins can be measured by specific assays for each particular protein that are known to those of skill in the art.

Long-term in vivo expression of a variety of proteins can be effected by the methods of the invention, preferably by in vivo administration of high titer retroviral vectors as described herein. A large variety of different proteins can be expressed for therapeutic applications in a number of different disease states. Preferred proteins include, cytokines and immune system modulators, hormones, growth factors, vaccine antigens, and proteins for treatment of inherited diseases.

Genes encoding any of the cytokine and immunomodulatory proteins described herein can be expressed in a gene delivery vehicle to achieve long term in vivo expression. Other forms of these cytokines which are known to those of skill in the art can also be used. For instance, nucleic acid sequences encoding native IL-2 and gamma-interferon can be obtained as described in U.S. Pat. Nos. 4,738,927 and 5,326,859, respectively, while useful muteins of these proteins can be obtained as described in U.S. Pat. No. 4,853,332. As an additional example, nucleic acid sequences encoding the short and long forms of mCSF can be obtained as described in U.S. Pat. Nos. 4, 847,201 and 4,879,227, respectively. Retroviral vectors expressing cytokine or immunomodulatory genes can be produced as described herein and in PCT publication number US 94/02951 entitled “Compositions and Methods for Cancer Immunotherapy”.

Gene delivery vehicles producing a variety of known polypeptide hormones and growth factors can be used in the methods of the invention to produce therapeutic long-term expression of these proteins. A large variety of hormones, growth factors and other proteins which are useful for long term expression by the retroviral vectors of the invention are described, for instance, in EP publication number 0437478B1, entitled “Cyclodextrin-Peptide Complexes.” Nucleic acid sequences encoding a variety of hormones can be used, including human growth hormone, insulin, calcitonin, prolactin, follicle stimulating hormone, leutinizing hormone, human chorionic gonadotropin and thyroid stimulating hormone. Gene delivery vehicles expressing polypeptide hormones and growth factors can be prepared by methods known to those of skill in the art and as described herein. For instance, a retroviral vector expressing human growth hormone can be prepared as described in the Examples. As an additional example, nucleic acid sequences encoding different forms of human insulin can be isolated as described in European Patent Publications EP 026598 or 070632, and incorporated into gene delivery vehicles as described herein.

Any of the polypeptide growth factors described herein can also be administered therapeutically by long-term expression in vivo with a gene delivery vehicle. For instance, a variety of different forms of IGF-1 and IGF-2 growth factor polypeptides are also well known the art and can be incorporated into gene delivery vehicles for long term expression in vivo. See e.g., European Patent No. 0123228B1, grant published on Sep. 19, 1993, entitled “Hybrid DNA Synthesis of Mature Insulin-like Growth Factors.” As an additional example, the long term in vivo expression of different forms of fibroblast growth factor can also be effected by the methods of invention. See, eg. U.S. Pat. No. 5,464,774, issued Nov. 7, 1995; U.S. Pat. No. 5,155,214, and U.S. Pat. No. 4,994,559, for a description of different fibroblast growth factors.

There are a number of proteins useful for treatment of hereditary disorders that can be expressed in vivo by the methods of invention. Many genetic diseases caused by inheritance of defective genes result in the failure to produce normal gene products, for example, thalassemia, phenylketonuria, Lesch-Nyhan syndrome, severe combined immunodeficiency (SCID), hemophilia, A and B, cystic fibrosis, Duchenne's Muscular Dystrophy, inherited emphysema and familial hypercholesterolemia (Mulligan et al., Science 260:926, 1993; Anderson et al., Science 256:808, 1992; Friedman et al., Science 244:1275, 1989). Although genetic diseases may result in the absence of a gene product, endocrine disorders, such as diabetes and hypopituitarism, are caused by the inability of the gene to produce adequate levels of the appropriate hormone insulin and human growth hormone respectively.

Gene therapy by the methods of the invention is a powerful approach for treating these types of disorders. This therapy involves the introduction of normal recombinant genes into somatic cells so that new or missing proteins are produced inside the cells of a patient. A number of genetic diseases have been selected for treatment with gene therapy, including adenine deaminase deficiency, cystic fibrosis, α₁-antitrypsin deficiency, Gaucher's syndrome, as well as non-genetic diseases. As an example of the present invention, a gene delivery vehicle can be used to treat Gaucher disease. Gaucher disease is a genetic disorder that is characterized by the deficiency of the enzyme glucocerebrosidase. This enzyme deficiency leads to the accumulation of glucocerebroside in the lysosomes of all cells in the body. For a review see Science 256:794, 1992 and The Metabolic Basis of Inherited Disease, 6th ed., Scriver, et al., vol. 2, p. 1677.

As additional examples, long term expression of Factor VIII or Factor IX is useful for treatment of blood clotting disorders, such as hemophilia. Different forms of Factor VIII, such as the B domain deleted Factor VIII construct described in Example 2 herein can be used to produce gene delivery vehicles expressing Factor VIII for use in the methods of the invention. In addition to clotting factors, there are a number of proteins which can be expressed in the gene delivery vehicles of the invention and which are useful for treatment of hereditary diseases. These include lactase for treatment of hereditary lactose intolerance, AD for treatment of ADA deficiency, and alpha-1 antitypsin for treatment of alpha-1 antitrypsin deficiency. See F. D. Ledley, J. Pediatics, 110:157-174, 1987; I. Verma, Scientific American: 68-84, Nov., 1987; and PCT Publication WO 9527512 entitled “Gene Therapy Treatment for a Variety of Diseases and Disorders” for a description of gene therapy treatment of genetic diseases.

There are a variety of other proteins of therapeutic interest that can be expressed in vivo by gene delivery vehicles using the methods of the invention. For instance sustained in vivo expression of tissue factor inhibitory protein (TFPI) is useful for treatment of conditions including sepsis and DIC and in preventing reperfusion injury. (See PCT Patent Publications Nos. WO 93/24143 ,WO 93/25230 and WO 96/06637.) Nucleic acid sequences encoding various forms of TFPI can be obtained, for example, as described in U.S. Pat. Nos. 4,966,852; 5,106,833; and 5,466,783, and can be incorporated in gene delivery vehicles as is described herein.

Other proteins of therapeutic interest such as erythropoietin (EPO) and leptin can also be expressed in vivo by gene delivery vehicles according to the methods of the invention. For instance EPO is useful in gene therapy treatment of a variety of disorders including anemia (see PCT publication number WO 9513376 entitled “Gene Therapy for Treatment of Anemia.”) Sustained gene therapy delivery of leptin by the methods of the invention is useful in treatment of obesity. (See WO 9605309 entitled “Obesity Polypeptides able to modulate body weight” for a description of the leptin gene and its use in the treatment of obesity.) Gene delivery vehicles expressing EPO or leptin can readily be produced using the methods described herein and the constructs described in these two patent publications.

A variety of other disorders can also be treated by the methods of the invention. For example, sustained in vivo systemic production of apolipoprotein E or apolipoprotein A by the gene delivery vehicles of this invention can be used for treatment of hyperlipidemia. (See J. Breslow et al., Biotechnology 12:365, 1994.) In addition, sustained production of angiotensin receptor inhibitor (T. L. Goodfriend et al., N. Engl. J. Med. 334:1469, 1996) can effected by the gene therapy methods described herein. As yet an additional example, the long term in vivo systemic production of angiostatin by the gene delivery vehicles of the invention is useful in the treatment of a variety of tumors. (See M. S. O'Reilly et al., Nature Med. 2:689, 1996).

5. Routes and Methods of Administration

A wide variety of routes and methods may be utilized in order to administer the gene delivery vehicles of the present invention. For example, intravenous (IV) administration can occur under a variety of protocols known to those of skill in the art. For instance, gene delivery vectors can be formulated for IV administration and administered as a single injection. Alternatively, the gene delivery vehicles can be delivered in a multiple injection protocol. An example of a multiple injection protocol is administration for three times a day for several consecutive days or on alternate days. The multiple injection schedule can be carried out over a number of days for example a week or 10 days or two weeks. The injection schedule can also be repeated. The total number of vector particles delivered can dispersed in varying amounts of formulation buffer. Depending on the volume delivered, the retroviral vectors can be delivered as an injection or as an IV formulation such as an IV drip which can be delivered over a longer period of time. Similarly, the rate of administration can vary. Details of the administration protocol such as the single or multiple injection schedule and volume and time of delivery can be determined experimentally by those of skill in the art, and will also vary depending on the particular gene of interest to be delivered. IV administration is a preferred route of administration for retroviral vectors expressing secretory proteins such as Factor VIII and human growth hormone.

Oral administration is easy and convenient, economical (no sterility required), safe (over dosage can be treated in most cases), and permits controlled release of the active ingredient of the composition (the recombinant retrovirus). Conversely, there may be local irritation such as nausea, vomiting or diarrhea, erratic absorption for poorly soluble drugs, and the recombinant retrovirus will be subject to “first pass effect” by hepatic metabolism and gastric acid and enzymatic degradation. Further, there can be slow onset of action, efficient plasma levels may not be reached, a patient's cooperation is required, and food can affect absorption. Preferred embodiments of the present invention include the oral administration of recombinant retroviruses that express genes encoding erythropoietin, insulin, GM-CSF cytokines, various polypeptides or peptide hormones, their agonists or antagonists, where these hormones can be derived from tissues such as the pituitary, hypothalamus, kidney, endothelial cells, liver, pancreas, bone, hemopoetic marrow, and adrenal. Such polypeptides can be used for induction of growth, regression of tissue, suppression of immune responses, apoptosis, gene expression, blocking receptor-ligand interaction, immune responses and can be treatment for certain anemias, diabetes, infections, high blood pressure, abnormal blood chemistry or chemistries (e.g., elevated blood cholesterol, deficiency of blood clotting factors, elevated LDL with lowered HDL), levels of Alzheimer associated amaloid protein, bone erosion/calcium deposition, and controlling levels of various metabolites such as steroid hormones, purines, and pyrimidines. Preferably, the recombinant retroviruses are first lyophilized, then filled into capsules and administered.

Buccal/sublingual administration is a convenient method of administration that provides rapid onset of action of the active component(s) of the composition, and avoids first pass metabolism. Thus, there is no gastric acid or enzymatic degradation, and the absorption of recombinant retroviruses is feasible. There is high bioavailability, and virtually immediate cessation of treatment is possible. Conversely, such administration is limited to relatively low dosages (typically about 10-15 mg), and there can be no simultaneous eating, drinking or swallowing. Preferred embodiments of the present invention include the buccal/sublingual administration of recombinant retroviruses that contain genes encoding self and/or foreign MHC, or immune modulators, for the treatment of oral cancer; the treatment of Sjogren's syndrome via the buccal/sublingual administration of such recombinant retroviruses that contain IgA or IgE antisense genes; and, the treatment of gingivitis and periodontitis via the buccal/sublingual administration of IgG or cytokine antisense genes.

Rectal administration provides a negligible first pass metabolism effect (there is a good blood/lymph vessel supply, and absorbed materials drain directly into the inferior vena cava), and the method is suitable of children, patients with emesis, and the unconscious. The method avoids gastric acid and enzymatic degradation, and the ionization of a composition will not change because the rectal fluid has no buffer capacity (pH 6.8; charged compositions absorb best). Conversely, there may be slow, poor or erratic absorption, irritation, degradation by bacterial flora, and there is a small absorption surface (about 0.05m²). Further, lipidophilic and water soluble compounds are preferred for absorption by the rectal mucosa, and absorption enhancers (e.g., salts, EDTA, NSAID) may be necessary. Preferred embodiments of the present invention include the rectal administration of recombinant retroviruses that contain genes encoding colon cancer antigens, self and/or foreign MHC, or immune modulators.

Nasal administration avoids first pass metabolism, and gastric acid and enzymatic degradation, and is convenient. In a preferred embodiment, nasal administration is useful for recombinant retrovirus administration wherein the recombinant retrovirus is capable of expressing a polypeptide with properties as described herein. Conversely, such administration can cause local irritation, and absorption can be dependent upon the state of the nasal mucosa.

Pulmonary administration also avoids first pass metabolism, and gastric acid and enzymatic degradation, and is convenient. Further, pulmonary administration permits localized actions that minimize systemic side effects and the dosage required for effectiveness, and there can be rapid onset of action and self-medication. Conversely, at times only a small portion of the administered composition reaches the brochioli/alveoli, there can be local irritation, and overdosing is possible. Further, patient cooperation and understanding is preferred, and the propellant for dosing may have toxic effects. Preferred embodiments of the present invention include the pulmonary administration of recombinant retroviruses that express genes encoding IgA or IgE for the treatment of conditions such as asthma, hay fever, allergic alveolitis or fibrosing alveolitis, the CFTR gene for the treatment of cystic fibrosis, and protease and collagenase inhibitors such as α-1-antitrypsin for the treatment of emphysema. Alternatively, many of the same types of polypeptides or peptides listed above for oral administration may be used.

Ophthalmic administration provides local action, and permit prolonged action where the administration is via inserts. Further, avoids first pass metabolism, and gastric acid and enzymatic degradation, and permits self-administration via the use of eye-drops or contact lens-like inserts. Conversely, the administration is not always efficient, because the administration induces tearing. Preferred embodiments of the present invention include the ophthalmic administration of recombinant retroviruses that express genes encoding IgA or IgE for the treatment of hay fever conjunctivitis or vernal and atomic conjunctivitis; and ophthalmic administration of recombinant retroviruses that contain genes encoding melanoma specific antigens (such as high molecular weight-melanoma associated antigen), self and/or foreign MHC, or immune modulators.

Transdermal administration permits rapid cessation of treatment and prolonged action leading to good compliance. Further, local treatment is possible, and avoids first pass metabolism, and gastric acid and enzymatic degradation. Conversely, such administration may cause local irritation, is particularly susceptible to tolerance development, and is typically not preferred for highly potent compositions. Preferred embodiments of the present invention include the transdermal administration of recombinant retroviruses that express genes encoding IgA or IgE for the treatment of conditions such as atopic dermatitis and other skin allergies; and transdermal administration of recombinant retroviruses encoding genes encoding melanoma specific antigens (such as high molecular weight-melanoma associated antigen), self and/or foreign MHC, or immune modulators.

Vaginal administration provides local treatment and one preferred route for hormonal administration. Further, such administration avoids first pass metabolism, and gastric acid and enzymatic degradation, and is preferred for administration of compositions wherein the recombinant retroviruses express peptides. Preferred embodiments of the present invention include the vaginal administration of recombinant retroviruses that express genes encoding self and/or foreign MHC, or immune modulators. Other preferred embodiments include the vaginal administration of genes encoding the components of sperm such as histone, flagellin, etc., to promote the production of sperm-specific antibodies and thereby prevent pregnancy. This effect may be reversed, and/or pregnancy in some women may be enhanced, by delivering recombinant retroviruses vectors encoding immunoglobulin antisense genes, which genes interfere with the production of sperm-specific antibodies.

Intravesical administration permits local treatment for urogenital problems, avoiding systemic side effects and avoiding first pass metabolism, and gastric acid and enzymatic degradation. Conversely, the method requires urethral catheterization and requires a highly skilled staff. Preferred embodiments of the present invention include intravesical administration of recombinant retrovirus encoding antitumor genes such as a prodrug activation gene such thymidine kinase or various immunomodulatory molecules such as cytokines.

Endoscopic retrograde cystopancreatography (ERCP) (goes through the mouth; does not require piercing of the skin) takes advantage of extended gastroscopy, and permits selective access to the biliary tract and the pancreatic duct. Conversely, the method requires a highly skilled staff, and is unpleasant for the patient.

Many of the routes of administration described herein (e.g., into the CSF, into bone marrow, into joints, intravenous, intra-arterial, intracranial intramuscular, subcutaneous, into various organs, intra-tumor, into the interstitial spaces, intra-peritoneal, intralymphatic, or into a capillary bed) may be accomplished simply by direct administration using a needle, catheter or related device.

Gene delivery vehicles can also be delivered to a target from outside of the body (as an outpatient procedure) or as a surgical procedure, where the vector is administered as part of a procedure with other purposes, or as a procedure designed expressly to administer the vector. Other routes and methods for administration include the non-pareneral routes as well as administration via multiple sites.

The gene delivery vehicles of the invention can also be delivered in ex vivo protocols. Ex vivo gene therapy protocols include those in which cells are removed from a patient, transduced in vitro and returned to the patient. Ex vivo gene therapy also encompasses protocols involving administration of producer cell lines capable of delivering a viral vector to a patient. See U.S. Pat. No. 5,399,346, U.S. Pat. No. 5,529,774, EP 476,953 and WO 96/33282 for a description of ex vivo gene therapy administration methods.

H. Formulation and Administration of Growth Factors

As is desribed herein, gene delivery vehicles of the present invention can be administered after induction of cell proliferation by a growth factor, or may be co-adminstered with a growth factor. The growth factors of the invention are administered by parenteral, topical, oral or by local administration. For example, the growth factors are adminstered parenterally, e.g., intravenously, subcutaneously, intradermally, or intramuscularly. Preferably, the growth factors are administered intravenously. Administration of the therapeutic agent of the invention can be accomplished by, for example, injection, catheterization, laser-created perfusion channels, cannulization, a particle gun, and a pump.

The growth factors of the invention are typically adminstered with a pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in REMINGTON'S PHARMACEUTICAL SCIENCES (Mack Pub. Co., N.J. 1991). Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier. The term “liposomes” refers to, for example, the liposome compositions described in U.S. Pat. No. 5,422,120, WO 95/13796, WO 94/23697, WO 91/14445 and EP 524,968 B1. Liposomes may be pharmaceutical carriers for the polypeptides of the invention.

The growth factors of the invention are administered in therapeutically effective amounts. The term “therapeutically effective amount” as used herein and applied to polypeptide growth factros refers to an amount of a growth factor that is capable of stimulating cell division in a target tissue in vivo. Stimulation of cell proliferation in a target tissue means that the number of dividing cells in the target tissue is greater than in the absence of treatment. The precise effective amount for a subject will depend upon the subject's size and health, the nature and extent of the condition being treated, recommendations of the treating physician, and particular growth factor that is used. The effective amount for a given situation can be determined by routine experimentation and will vary from growth factor to growth factor. For example, for HGF, a dose of 1 ug/kg to 2 mg/kg body weight, and more preferably from 10 ug/kg to 200 ug/kg body weight is used. In the case of KGF, a dose of 100 ug/kg to 5mg/kg body weight, or more preferably a dose of 1 mg/kg to 50 mg/kg body weight is used. Dose amounts for the other growth factors used in the claimed methods are known to those of skill in the art or can readily be determined experimentally.

Clofibrate, or the other proxisome proliferators, can be administered by IP injection (5-500 mg/kg), or orally (5-500 mg/kg). More preferably the dosages are 10-100 mg/kg. A typical dosing schedule is daily administration for 3-10 days. A tapered dosing can alternatively be employed. Following clofibrate dosing, retroviral vectors can be administered, preferably intravenously, at doses ranging from 1E5 to 1E 11 cfu per injection. Injection schedules of one to three times daily, for one to ten days, will be employed. Repeat administrations of retroviral vector with or without repeat clofibrate or growth factor dosing can be performed.

The following examples are offered by way of illustration, and not by way of limitation.

### EXAMPLES

### Example 1

### PREPARATION OF RETROVIRAL BACKBONES

A. Preparation of Retroviral Backbones Kt-1

The Moloney murine leukemia virus (MOMLV) 5′ long terminal repeat (LTR) EcoR I-EcoR I fragment, including gag sequences, from the N2 (Armentano et al., J Vir. 61:1647, 1987; Eglitias et al., Science 230:1395, 1985) vector was ligated into the plasmid SK⁺ (Stratagene, San Diego, Calif.). The resulting construct was designated N2R5. The N2R5 construct was mutated by site-directed in vitro mutagenesis to change the ATG start codon to ATT, preventing gag expression. This mutagenized fragment is 200 base pairs (bp) in length and flanked by Pst I restriction sites. The Pst I-Pst I mutated fragment was purified from the SK⁺ plasmid and inserted into the Pst I site of N2 MoMLV 5′ LTR in plasmid pUC31 to replace the non-mutated 200 bp fragment. The plasmid pUC31 is essentially pUC19 (Stratagene, San Diego, Calif.), except that additional restriction sites Xho I, Bgl II, BssH II and Nco I were inserted between the EcoR I and Sac I sites of the polylinker. This construct was designated pUC31/N2R5gM.

A 1.0 kilobase (Kb) MoMLV 3′ LTR EcoR I-EcoR I fragment from N2 was cloned into plasmid SK⁺ resulting in a construct designated N2R3⁻. A 1.0 Kb Cla I-Hind III fragment was purified from this construct.

The Cla I-Cla I dominant selectable marker gene fragment from pAFVXM retroviral vector, comprising a SV40 early promoter driving expression of the neomycin (neo) phosphotransferase gene, was cloned into the SK⁺ plasmid. This construct was designated SV⁺SV₂-neo. A 1.3 Kb Cla I-BstB I gene fragment is purified from the SK⁺SV₂-neo plasmid.

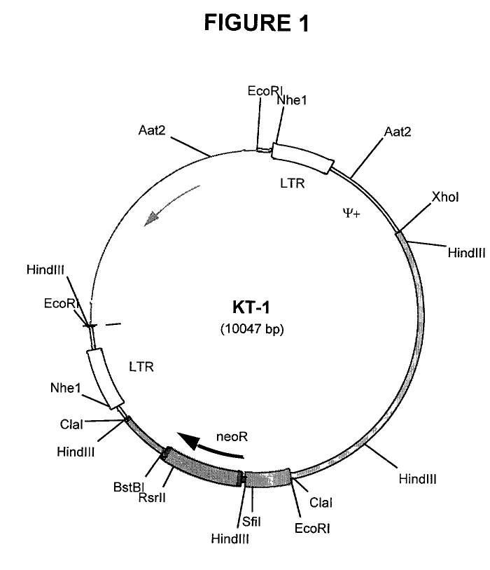

KT-1 vector was constructed by a three part ligation in which the Xho I-Cla I fragment containing the gene of interest and the neo gene under the control of the SV40 promoter/enhancer and the 1.0 Kb MoMLV 3′ LTR Cla I-Hind III fragment are inserted into the Xho I-Hind III site of pUC31/N2R5gM plasmid (FIG. 1).

B. Preparation of Retroviral Backbone pBA5b

1. Preparation of a retroviral vector construct that does not contain an extended packaging sequence (Ψ)

This example describes the construction of a retroviral vector construct using site-specific mutagenesis. Within this example, a MoMLV retroviral vector construct is prepared wherein the packaging signal “Ψ” of the retroviral vector is terminated at basepair 617 of SEQ ID NO: 1, thereby eliminating the ATG start of gag.

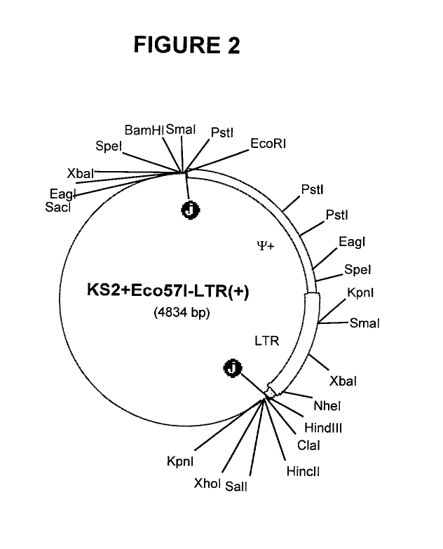

Briefly, pMLV-K (Miller, J. Virol 49:214-222, 1984-an infectious clone derived from pMLV-1 Shinnick et al., Nature 293:543-548, 1981) was digested with Eco57I, and a 1.9kb fragment is isolated. (Eco571 cuts upstream from the 3′ LTR, thereby removing all env coding segments from the retroviral vector construct.) The fragment was then blunt ended with T4 polymerase (New England Biolabs), and all four deoxynucleotides, and cloned into the EcoRV site of phagemid pBluescript II KS+ (Stratagene, San Diego, Calif.). This procedure yields pKS2+Eco57I-LTR(+) (FIG. 2), which was screened by restriction analysis. When the (+) single stranded phagemid was generated, the sense sequence of MoMLV was isolated.

2. Substitution of Nonsense Codons in the Extended Packaging Sequence (Ψ+)

This example describes modification of the extended packaging signal (Ψ+) by site-specific mutagenesis. In particular, the modification substitutes a stop codon, TAA, at the normal ATG start site of gag (position 631-633 of SEQ ID NO: 1), and an additional stop codon TAG at position 637-639 of SEQ ID NO: 1.

Briefly, an Eco57I -EcoRI fragment (MOMLV basepairs 7770 to approx. 1040) from pN2 (Amentano et al., J Virol. 61:1647-1650, 1987) was first cloned into pBluescript II KS+phagemid at the SacII and EcoRI sites (compatible). Single stranded phagemid containing antisense MoMLV sequence, was generated using helper phage M13K07 (Stratagene, San Diego, Calif.). The oligonucleotide 5′-CTG TAT TTG TCT GAG AAT TAA GGC TAG ACT GTT ACC AC (SEQ ID NO: 3) was synthesized, and utilized according to the method of Kunkel (PNAS 82:488, 1985), in order to modify the sequence within the Ψ region to encode stop codons at nucleotides 631-633 and 637-639.

3. Removal of Retroviral Packaging Sequence Downstream from the 3′ LTR

Retroviral packaging sequence which is downstream from the 3′ LTR was deleted essentially as described below. Briefly, pKS2+Eco57I-LTR(−) was digested with BalI and HincII, and religated excluding the BalI to HincII DNA which contains the packaging region of MoMLV.

4. Construction of Vector Backbones

Constructs prepared in sections B and C above, were combined with a plasmid vector as described below, in order to create a retroviral vector backbone containing all elements required in cis, and excluding all sequences of 8 nucleic acids or more contained in the retroviral portion of the gag-pol and env expression elements.

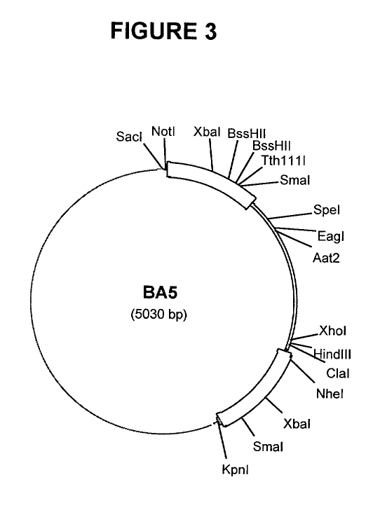

Briefly, parts B and C are combined as follows: The product of B was digested with NheI and EcoRi and a 1456 basepair fragment containing the LTR and modified Ψ+ region is isolated. The fragment is ligated into the product of C at the unique (compatible) restriction sites SpeI and EcoRI. The resultant construct was designated pBA5a (FIG. 3; see also U.S. Ser. No. 08/437,465).

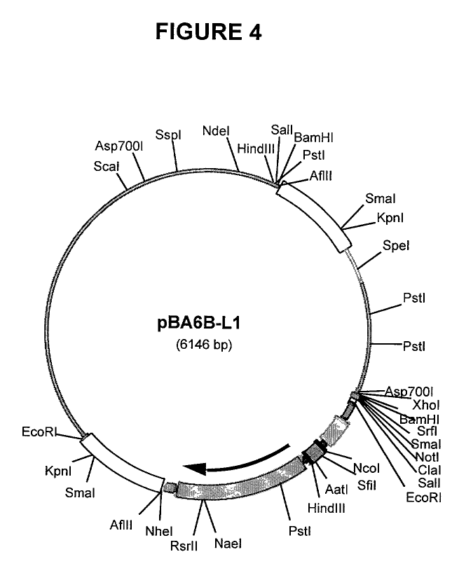

The vector pBA5a was cut with NotI and the end was made blunt by filling in the 5′ overhang with Klenow (Sambrook et al., Mol. Cloning, CSH, 1989) followed by digestion with EcoR I. The resulting insert was ligated to pUC 18 cut with Sma I and EcoR I to make pBA5b. The neo resistance marker gene was added by inserting the Xho I to BstB I fragment from KT-1 into pBA5b, digested with Xho I and Cla I, to make pBA6b. A polylinker was added by annealing two oligonucleotides: (1)-5′ TCGAGGATCG CGCCGGGCGG CCGCATCGAT GTCGACG (Sequence ID No. 4) and (2) 5′-CGCGTCGACA TCGATGCGGC CGCCCGGGCG GATCC (Sequence ID No. 5) and ligating the product to pBA6b cut with Xho I and Cla I to make pBA6bL1 (see FIG. 4).

### Example 2

### USE OF HUMAN BETA GALACTOSIDASE AS A GENETIC MARKER IN RETROVIRAL VECTOR

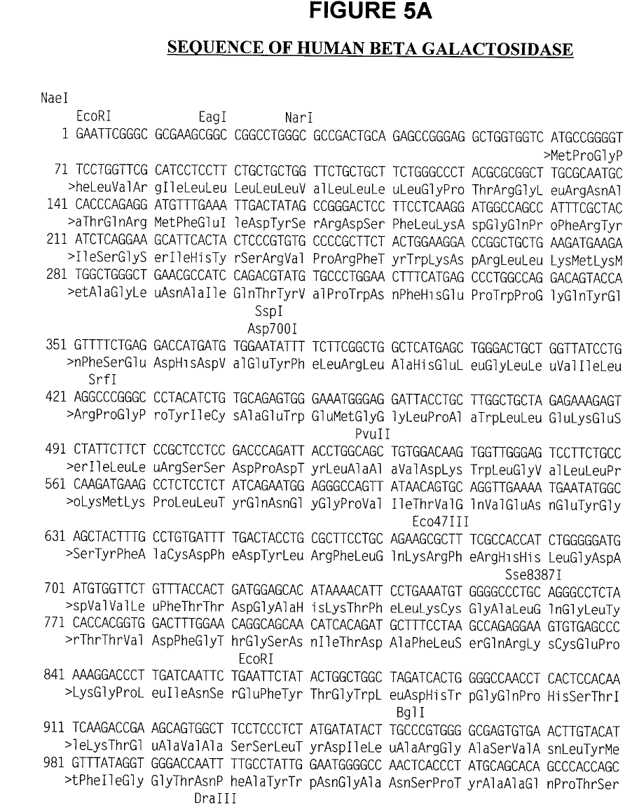

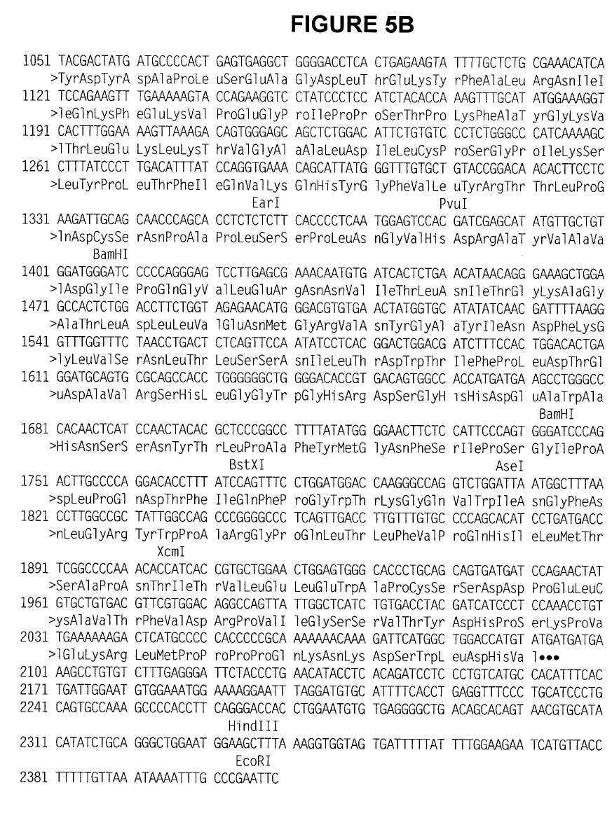

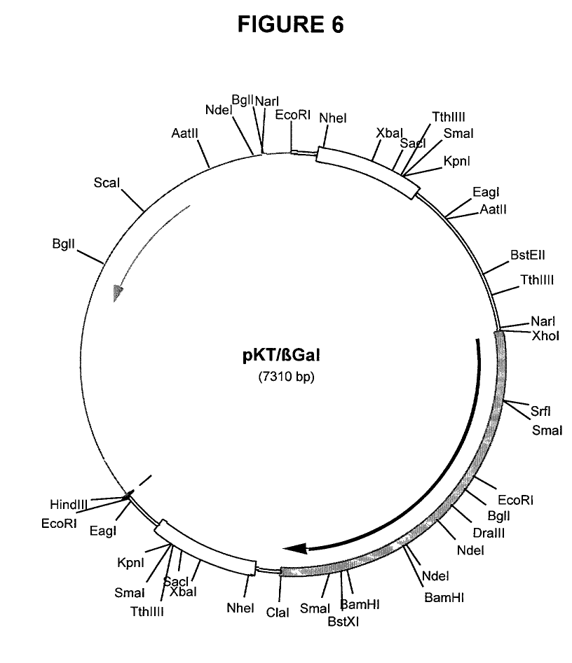

Human beta galactosidase mRNA is obtained from human liver tissue prepared with a MicroFastTrak™ kit (Invitrogen, San Diego, Calif.). The sequence of the cDNA for human beta galactosidase is listed in FIG. 5. This is used as a template for RT PCR reaction using the GeneAmp® RNA PCR kit (Perkin Elmer) and primers: 5′ GGG GGG CTC GAG ATG ACG CGC GGC TTG CGC AAT GC (Sequence ID No. 6) and 3′ GGG GGG ATC GAT TTC ATC ATC ATA CA (Sequence ID No. 7). The resulting 2.0 Kb human β-galactosidase cDNA, has no signal peptide, Xho I at the 5′ end, and Cla I at the 3′ end. It is inserted into the Moloney retroviral vector KT-1 at the Xho I and Cla I sites to make KT1/hβGal (FIG. 6). This removal of the signal peptide from human beta galactosidase converts it from microsomal to cytoplasmic in distribution which allows conversion of the prodrug conjugate to occur in the cytoplasm rather than in microsomes.

KT1/hβ3Gal is used to make a vector producing cell line by pseudotyping with VSV G protein (Bums, J. C. et al., PNAS 90:8033-8037, 1993). This method consists of cotransfection of 293 2-3 (Bums, J. C. et al., PNAS 90:8033-8037, 1993) with 10 μg of each of retroviral vector KT/hβGA1 with 10 μg of the VSV G protein vector, MLPG by CaPO4 transfection with the ProFection kit according to the manufacturer's instructions (Promega, Madison, Wis.). The CaPO₄-containing media is replaced with fresh DMEM/10% FBS after 16 hours then incubated overnight. The resulting culture supernatant containing VSV-G pseudotyped vector is filtered through 0.45 μm filter. This is used for transduction of the retroviral packaging cell line, DA (see PCT Publication No. WO 92/05266). These cells are subjected to cloning by limiting dilution, and the best clones selected by, e.g., PCR titering as described in Example 5. The supernatants of these cell lines are harvested, passed through 0.45 μm filters and stored at −80 C. in aliquots until use.

Supernatant from the selected vector producing DA/hβGa1 is used to transduce HT1080 target cells, which are then fixed and stained with Xgal (Irwin et al., J Virol. 68:50361994).

### Example 3

### USE OF HUMAN BETA GALACTOSIDASE FOR CONVERSION OF PRODRUG TO ACTIVE FORM FOR ABLATION THERAPY

The prodrug conjugate, N-[4-(β-D-galactopyranosyl) Benzyloxycarbonyl]-daunorubicin, is synthesized in a manner similar to that described in S. Andrianomenjanahary et al., Bioorganic & Medicinal Chemistry Letters 2:1093-1096, 1992, using the method of Danishevsky to generate the β-D-galactopyranoside (S. J. Danishevsky and M. T. Bilodeaux, Angewante Chemie Int'l Ed. English 35:1380-1419, 1996).

The relative sensitivity of HT1080 with and without hβGal to daunorubicin and N-[4-(β-D-galactopyranosyl)Benzyloxycarbonyl]-daunorubicin is measured as follows: HT 1080 cells are transduced with the DA/hβGal supernatant in 8 μg/ml polybrene overnight, then rinsed, fed fresh DMEM/10% FBS, and incubated overnight. The effect of daunorubicin and N-[4-(β-D-galactopyranosyl) Benzyloxycarbonyl]-daunorubicin is measured by plating 1×10⁴ cells per well in 96 well dishes of transduced and untransduced cells (P. D. Senter et al., PNAS 85:4842-4846, 1988). These are incubated for six hours in concentrations of daunorubicin, N-[4-(β-D-galactopyranosyl)Benzyloxycarbonyl]-daunorubicin (0 to 75 μM) or media alone. The wells are washed and incubated in media for 12 hours, then receive a pulse of [³H]thymidine (1 μCi/well) for six hours. The cells are transferred to glass fiber filters and counted in a scintillation counter (Beckman).

### Example 4

### USE OF HUMAN PLACENTAL ALKALINE PHOSPHATASE AS A GENETIC MARKER IN RETROVIRAL VECTOR

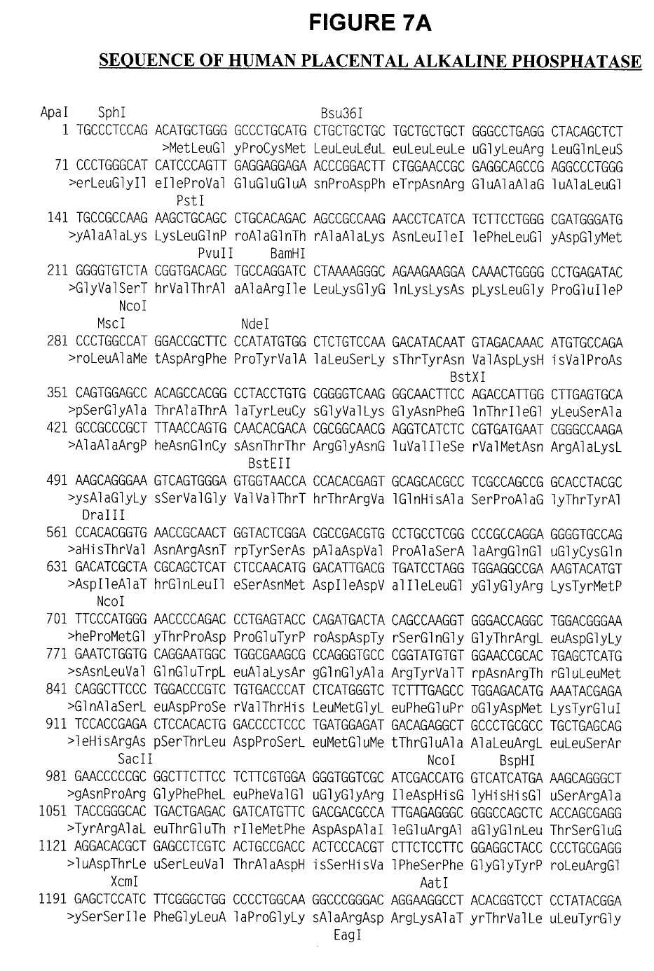

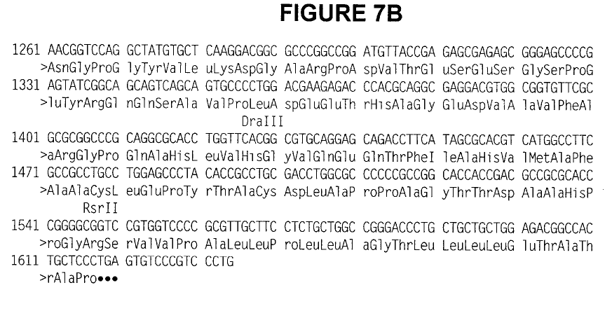

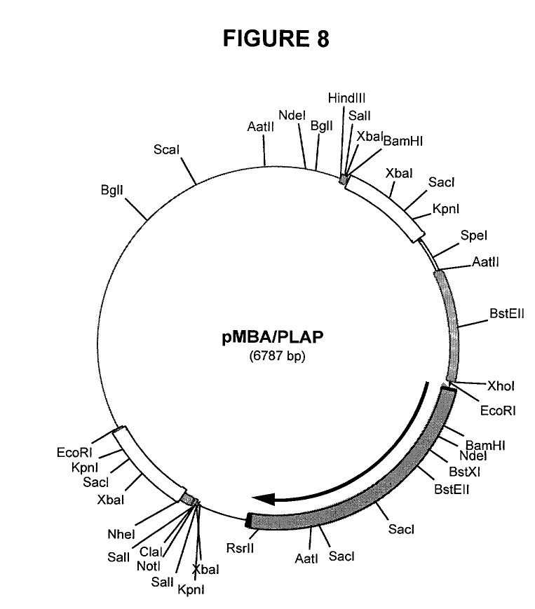

Human placental alkaline phosphatase cDNA (sequence shown in FIG. 7) was cloned from the vector pSVT7/PLAP (C. Hummer and J. L. Millan, Biochem. J. 274:91-95, 1991) into pCI (Promega, Madison, Wis.) at the EcoR I and Kpn I sites. This insert was then cut out of pCI with Xho I and Cla I and cloned into the Xho I and Cla I sites of the retroviral vector pMBA to make pMBA/hPLAP (FIG. 8).

MBA/hPLAP was used to make a vector producing cell line by pseudotyping with VSV G protein (Burns, J.C. et al., PNAS 90:8033-8037, 1993). This method consists of cotransfection of 293 2-3 with 10 μg of each of retroviral vector MBA/hPLAP with 10 μg of the VSV G protein vector, MLPG by CaPO₄ transfection with the ProFection kit according to the manufacturer's instructions (Promega, Madison, Wis.). Sixteen hours post-transfection the cells were rinsed and fed fresh DMEM/10% FBS. The media was removed after 24 hours of incubation and filtered through 0.45 μm syringe filter. This supernatant was applied to the packaging cell line, DA, with 8 μg/ml of polybrene.

The DA cells were selected by adsorption onto antibody-coated magnetic beads followed by exposure to a magnetized iron column (MACS) using the Miltenyi MiniMACS system (Miltenyi Biotec Inc., Sunnyvale, Calif.) as follows: the antibody, MIG-P1 (Biosource International, Camarillo, Calif.), specific for the placental alkaline phosphatase, was bound at a 1:50 dilution to 0.5 to 1×10⁷ transduced cells in 200 μl PBS/2% FBS on ice for 30 min. The goat polyclonal anti-mouse IgG magnetic beads (Miltenyi cat. #484-01) beads are washed by resuspending 200 μl in cold PBS then loading them on a Miltenyi column (Miltenyi cat. #422-01) held in the magnet. The beads are eluted by removing the column from the magnet and eluting in 200 μl PBS/2% FBS. The beads were then incubated with the antibody-treated transduced DA cells, the cells are collected by centrifugation 10 minutes at 1000 rpm, 4° C., and loaded on a fresh Miltenyi column on the magnet (prepared according to manufacturer's instructions). Following elution of the non-bound cells, the column was washed with cold PBS/2% FBS, and then removed from the magnet and the bound cells were then washed off the column with cold PBS/2% FBS. The cells were plated in DMEM/10% FBS and allowed to grow out. The percentage of positive cells was measured by FACS analysis using the same monoclonal antibody, MIG-P1, followed by staining with FITC-goat anti-mouse IgG (Fab′) fragment, and analysis on a Becton-Dickenson FACS analyzer. Supernatant from the DA/hPLAP cells was collected and filtered through 0.45 μm syringe filter and stored at −80 C. The cells are subjected to cloning by limiting dilution, and the best clones selected by, e.g., PCR titering or Fast Red staining as described in Example 5.

The relative sensitivity of HT1080 with and without hPLAP to etoposide and etoposide phosphate is measured as follows: Etoposide phosphate is generated by phosphorylation of etoposide (Bristol-Myers) using the method described in Senter et al., PNAS 85:4842-4846, 1988. HT 1080 cells are transduced with the DA/hPLAP supernatant in 8 μg/ml polybrene overnight, then rinsed, fed fresh DMEM/10% FBS, and incubated overnight. The effect of etoposide and etoposide phosphate is measured by plating 1×10⁴ cells per well in 96 well dishes of transduced and untransduced cells. These are incubated for six hours in concentrations of etoposide, etoposide phosphate (0 to 75 μM) or media alone. The wells are washed and incubated in media for 12 hours, then receive a pulse of [³H]thymidine (1 μCi/well) for six hours. The cells are transferred to glass fiber filters and counted in a scintillation counter (Beckman).

### Example 5

### PRODUCER CELL TITERING METHODS

A. Titering of Vector Via Pcr Amplification Using Vector Specific Primers.

Vector titer can be determined in a PCR assay by correlation of detected provector sequences in transduced cells to a vector standard run in parallel. Briefly, both vector test sample and vector standard are used to transduce target cells (e.g., HT1080) in parallel (using serial dilutions of both vector test sample and vector standard), and specific DNA sequences of the provectors integrated in the target cells are amplified via PCR. The PCR primers amplify a desired fragment within the vector LTR. The amount of amplified PCR amplicons of the vector test sample is then correlated to the PCR amplicons of the vector standard and expressed as colony forming unit equivalents (cfu-eq). PCR amplicons can be detected via incorporation of radiolabel during the PCR reaction. Radiolabel signals are quantitated using a phosphor imaging system.

For example, HT1080 cells are plated in 6 well plates at 3e5 cells per well. Twenty-four hours later, transductions are performed with the DA Cbβgal vector at 3e5 to 1e4 cfu per well. Vector test sample used for transductions is diluted 1/10 to 1/1000 (depending on the expected titer) to achieve transductions within the range of the assay. DNA is isolated 72 hours after transduction via phenol/chloroform extraction and ethanol precipitation and quantitated via microfluorometry using Hoechst dye 33258.

Genomic DNA (175ng per PCR reaction) is then amplified in a 50 μl PCR reaction containing 2 mM MgCl₂, 0.2 mM DATP, dCTP, TTP, and dGTP, 50 mM KCl, 10 mM Tris/HCl (pH 8.3), 0.4 mM of each primer F2A and R2A, 1.25 units of Amplitaq™ DNA polymerase (previously incubated with Taqstart™ antibody in IX Taqstart™ buffer (Clonetech, Palo Alto Calif.), and 0.2 μCi of Redivue [α-³²P] dCTP. Five microliters from each PCR reaction are blotted onto DE81 Ion Exchange Chromatography Paper (Whatman, Maidstone England) and washed 3 times with a phosphate buffered wash solution. The signals on the membrane are quantitated using a phosphor imager. A standard curve is generated by plotting the PCR signals versus the cfu-eq of the vector standard. The straight line equation is used to extrapolate the cfu-equivalent titer of the test samples.

Primer sequences:

(Sequence ID No. 29)
F2A primer: 5′ CTGTAGGTTTGGCAAGCTAGC 3′

B. Fast Red Staining of Adherent PLAP Cells

This assay can be utilized to detect the presence of adherent PLAP cells and hence, can be used to titer PLAP producer lines. Briefly, media is drained from plates containing adherent PLAP cells. One milliliter of fixing solution (PBS +2% formaldehyde +0.2% glutaraldehyde ) is added per well and allowed to incubate for 5 minutes at room temperature. The fixing solution is aspirated and the cells are washed with 2 mls of PBS. The wells are aspirated once more and the plates are incubated at 56° C. (with humidity) for 20 min. One milliliter of freshly prepared Fast Red Stain (TR/Naphthol AS_MX Tablet Set, Sigma) is added to each well and the plates are allowed to incubate at room temperature from 2 hours to overnight. The percent transduced/transfected cells is determined by counting red and non-red cells.

### Example 6

### DEMONSTRATION OF FUNCTION OF hPLAP IN ERADICATING TUMOR GROWTH IN NUDE MICE

hPLAP is able to convert the prodrugs, mitomycin phosphate (MOP) and etoposide phosphate (EP), into an active mitomycin C derivative, mitomycin alcohol, and etoposide. 5e5 HT1080 cells or HT/hPLAP cells (HT1080 cells stably expressing hPLAP) are inoculated subcutaneously into nude mice (Balb/c). Tumor development occurs in 7-14 days. Etoposide phosphate is prepared by the method of Senter et al., Cancer Res. 49:5789-92, 1988, or obtained from a pharmacy (e.g., manufactured by Bristol-Myers Squibb). Animals are dosed with EP as described (Senter et al., 1988) 2-10 days after inoculation with cells. Control animals inoculated with parental HT1080 cells develop tumors rapidly that are resistant to the effect of EP. However animals inoculated with HT/hPLAP cells demonstrate a dose-dependent reduction in tumor growth after administration of EP. Experiments involving injection of HT1080 cells in one flank and HT/hPLAP cells in the contralateral flank demonstrate that the EP effect is specific for cells expressing HPLAP.

### Example 7

### USE OF HUMAN CYTOCHROME P-450 2B FOR CONVERSION OF PRODRUG TO ACTIVE FORM FOR ABLATION THERAPY

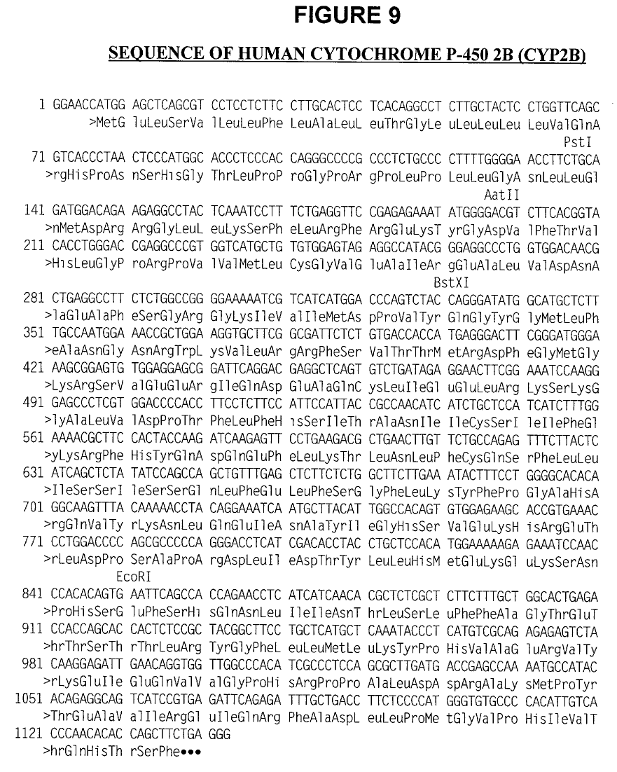

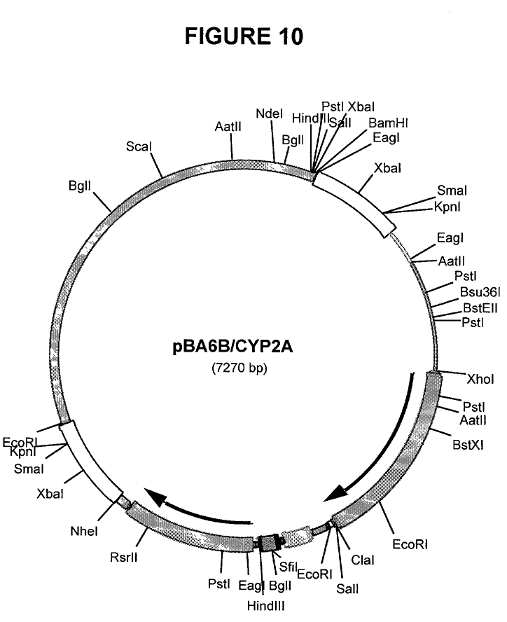

Human cytochrome P-450 2B mRNA is obtained from human liver tissue prepared with a MicroFastTrak™ kit (Invitrogen, San Diego, Calif.). The sequence of the CDNA for human cytochrome P-450 2B is listed in FIG. 9. This is used as a template for RT PCR reaction using the GeneAmp® RNA PCR kit (Perkin Elmer) and primers: 5′ GGG GGG CTC GAG GGC ACC ATG GAG CTC AG (Sequence ID No. 8) and 3′ GGG GGG ATC GAT CCC TCA GAA GCT GGT GTG (Sequence ID No. 9). The resulting 1.13 Kb human cytochrome P-450 2B cDNA has xho I at the 5′ end and Cla I at the 3′ end, and is inserted into the Moloney retroviral vector pBA6BL1 at the Xho I and Cla I sites to make BA6/CYP2B (FIG. 10).

BA6/CYP2B is used to make a vector producing cell line by pseudotyping with VSV G protein (Bums, J. C. et al., PNAS 90:8033-8037, 1993) as described in Example 2. Briefly, 10 μg of each of retroviral vector BA6/CYP2B with 10 μg of the VSV G protein vector, MLPG introduced into 293 2-3 cells by CaPO₄ transfection with the ProFection kit according to the manufacturer's instructions (Promega, Madison, Wis.). The CaPO₄-containing media is replaced with fresh DMEM/10% FBS after 16 hours then incubated overnight. The resulting culture supernatant containing VSV-G pseudotyped vector is filtered through 0.45 μm filter. This is used for transduction of the retroviral packaging cell line, DA. The cells are then subjected to cloning by limiting dilution, and the best clones selected by, e.g., PCR titering or Fast Red staining as described in Example 5. Cells are then grown to confluency, and the supernatants of these cell lines were harvested, passed through 0.45 μm filters and stored at −80 C. in aliquots until use.

The relative sensitivity of HT1080 with and without CYP2B to cyclophosphamide is measured as follows: HT 1080 cells are transduced with the DA/CYP2B supernatant in 8 μg/ml polybrene overnight, then rinsed, fed fresh DMEM/10% FBS, and incubated overnight. The effect of cyclophosphamide is measured by plating 1×10⁴ cells per well in 96 well dishes of transduced and untransduced cells. These are incubated for six hours in concentrations cyclophosphamide (0 to 1000 μM) or media alone. The wells are washed and incubated in media for 12 hours, then receive a pulse of [³H]thymidine (1 μCi/well) for six hours. The cells are transferred to glass fiber filters and counted in a scintillation counter (Beckman).

### Example 8

Use of Furin as a Cell-Bound Prodrug Convertase for Ablation Therapy

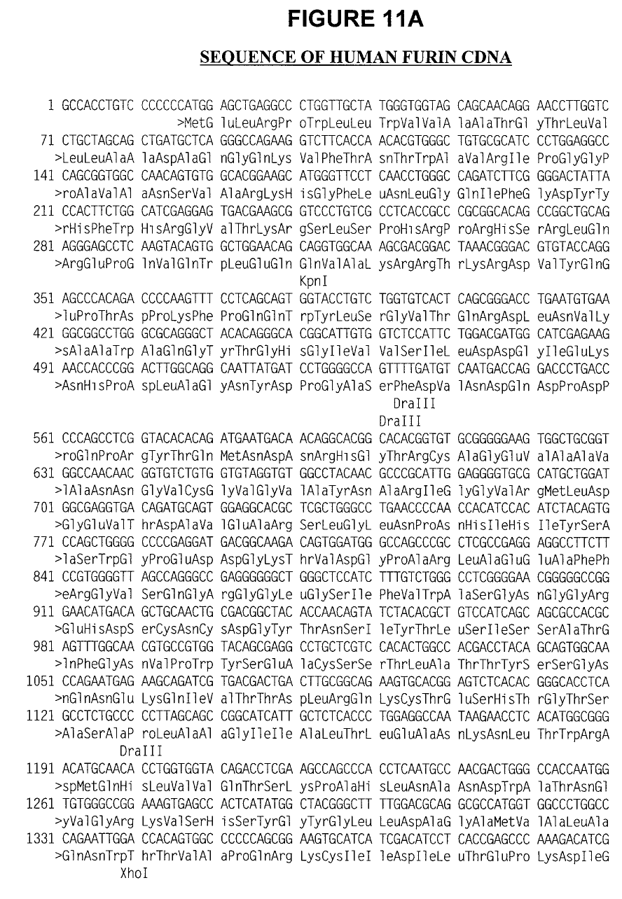

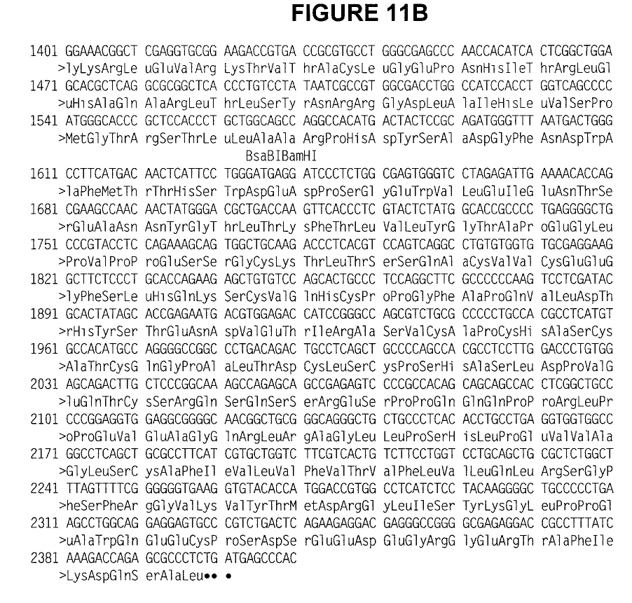

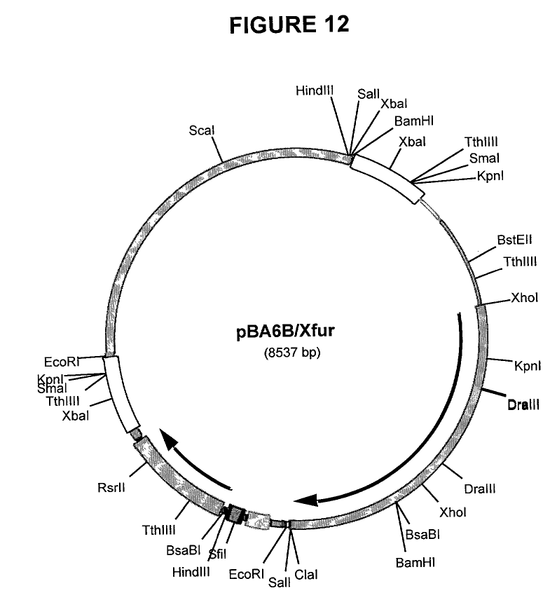

cDNA encoding furin is made by RT PCR using mRNA prepared by FastTrak™ (Invitrogen, San Diego) from human cell line HT1080 as a template. The primers (5′ flanking: 5′ CCC CCC CTC GAG ACC TGT CCC CCC CAT GGA G (Sequence ID No. 10), and 3′ flanking: 5′ CCC CCC ATC GAT GTG GGC TCA CAG AGG GCG C (Sequence ID No. 11)) are used in RT PCR reaction with the GeneAmp kit from Perkin Elmer according to manufacturer's instructions. The resulting PCR product is cloned into the TA vector using the TA cloning kit (Invitrogen) and is verified by DNA sequence analysis. An alteration is made in the cytosolic domain of furin to alter the distribution from trans-Golgi to cell surface localization, by deletion of the acidic cluster from residue 766 to 783 (FIG. 11) using overlap PCR with the flanking primers above and the deletion primers (5′ del: 5′ ATA AAG GCG GTC CTT TCA GGG GGC AGC CCC TTC TA (Sequence ID No. 12) and 3′ del: 5′ GGG GCT GCC CCC TGA AAG GAC CGC CTT TAT CAA AG (Sequence ID No. 13) in the following PCR reactions: The 5′ flanking and 5′ del primers in one tube and the 3′ flanking and 3′ del primers in another, both using the furin construct as template. The resulting left (2.3 Kb) and right (60 bp) bands are purified by agarose gel electrophoresis and the DNA is purified by GeneClean™ kit (Bio101, San Diego, Calif.). The two fragments are used as templates in a PCR reaction with the 5′ and 3′ flanking primers. The resulting PCR product with Xho I and Cla I at the 5′ and 3′ termini respectively is cloned into the vector pBA6BL1 at the Xho I and Cla I sites to make pBA6/Xfur (FIG. 12). The DNA sequence is verified by automated DNA sequencing methodology (Perkin-Elmer).

BA6/Xfur is used to make a vector producing cell line by pseudotyping with VSV G protein (Burns, J. C. et al., PNAS 90:8033-8037, 1993) as described in Example 2. Briefly, 10 μg of each of retroviral vector BA6/Xfur with 10 μg of the VSV G protein vector, MLPG introduced into 293 2-3 cells by CaPO₄ transfection with the ProFection kit according to the manufacturer's instructions (Promega, Madison, WI). The CaPO₄-containing media is replaced with fresh DMEM/10% FBS after 16 hours then incubated overnight. The resulting culture supernatant containing VSV-G pseudotyped vector is filtered through 0.45 μm filter. This is used for transduction of the retroviral packaging cell line, DA. The cells are subjected to cloning by limiting dilution, and the best clones selected by, e.g., PCR titering or Fast Red staining as described in Example 5. The supernatants of these cell lines were harvested, passed through 0.45 μm filters and stored at −80 C. in aliquots until use.

The prodrug substrate for Xfur is synthesized by standard Merrifield peptide synthetic methodology as Arg-Lys-Lys-Arg (Sequence ID No. 28) without deprotection. This is conjugated with phenylenediamine mustard (Everett, J. L. and Ross, W. C. J., J Chem. Soc.: 1972, 1949) in a mixed anhydride coupling (Chakravarty, P. K. et al., J. Med. Chem. 26:633-638, 1983) followed by deprotection with trifluoroacetic acid to make RKKR-phenylenediamine mustard.

The relative sensitivity of B 16 murine melanoma with and without Xfur to RKKR-phenylenediamine mustard in vitro is measured as follows: HT 1080 cells are transduced with the DA/Xfur supernatant in 8 μg/ml polybrene overnight, then rinsed, fed fresh DMEM/10% FBS, and incubated overnight. The effect RKKR-phenylenediamine mustard is measured by plating 1×10⁴ cells per well in 96 well dishes of transduced and untransduced cells. These are incubated for six hours in concentrations RKKR-phenylenediamine mustard (0 to 500 μM) or media alone. The cells are counted with trypan blue to determine viability and growth.

B 16 cells transduced with DA/Xfur and selected as described above. 1×10⁷ transduced and untransduced B16 cells are implanted subcutaneously in the left and right flanks of BALB/c mice respectively and allowed to establish palpable tumors. RKKR-phenylenediamine mustard from 0 to 8 mg/kg is injected daily into the peritoneum of mice on days 1 to 9 and the survival of the mice is used as a measure.

### Example 9

### VECTORS EXPRESSING DCK

A. Generation of KT1/dCK Vector

To generate a retroviral expression vector encoding the human dCK coding sequences, firstly, the dCK cDNA must be obtained. Briefly, cellular mRNA is isolated from human T-cell lines, MOLT-3 (ATCC CRL 1552), MOLT-4 or Jurkat cells using the MicroFastTrak™ kit (Invitrogen, San Diego, Calif.). The mRNA preparation is used as a template for RT PCR reaction using the GeneAmp® RNA PCR kit (Perkin Elmer) and primers: 5′ GGG GGG CTC GAG CCC CGA CAC CGC GGC GGG CCG (Sequence ID No. 14) and 3′ GGG GGG ATC GAT GCT GAA GTA TCT GGA ACC (Sequence ID NO. 15). The resulting 1.0 Kb human dCK cDNA has a Xho I at the 5′ end and Cla I at the 3′ end. It is inserted into the Moloney retroviral vector KT-1 at the Xho I and Cla I sites to make KT1/hdCK.

KT1/hdCK is used to make a VCL by pseudotyping with VSV G protein as described above. The relative sensitivity to cytosine arabinoside (ara-C) of 9L glioblastoma cells transduced with the KT/hdCK vector versus control cells transduced with KT1/beta-gal is evaluated as described below. 9L cells are transduced with vector supernatant from VCL specific for KT1/hdCK or KT1/β-gal. Cells are transduced with vector supernatant in the presence of 8 μg/ml polybrene overnight, rinsed and fed with fresh media and incubated overnight. The effect of dCK is measured by plating 2×103 cells/200 μl in individual wells of 96 well dishes. The cells are incubated for 12 hours and then treated with ara-C for 96 hours. The cells are fixed and stained with 0.05% Methylene blue. The dye is eluted with 0.33 M HCl for 15 minutes with agitation and absorbance measured in a microplate reader at 600 nm. Alternatively, the cells may be stained with Trypan blue and viable/dead cells evaluated.

B. Evaluation of the Effect of hdCK In Vivo

9L cells expressing hdCK or β-gal are injected into Fischer 344 rats and evaluated for their in vivo sensitivity to araC. One million stably transduced cells expressing hdCK or P-gal are injected intradermally into opposite flanks of adult rats. Small tumor nodules are evident between days 7-10. At day 9, animals are treated with ara C or PBS. The dose of ara C is 200 mg/kg every 8 hours for 2 days, followed by another dose of ara C 6 days later. Tumor volumes are measured periodically through the course of the experiment.

### Example 10

### GENERATION OF KT1/hENT1 VECTOR

To generate a retroviral expression vector encoding the human hENT1 coding sequences, first the hENT1 cDNA is obtained. Briefly, cellular mRNA is isolated from the acute myelogenous leukemia cell line KG-1 (ATCC CCL 246) using the MicroFastTrak™ kit (Invitrogen, San Diego, Calif.). The mRNA preparation is used as a template for RT PCR reaction using the GeneAmp® RNA PCR kit (Perkin Elmer) and primers as follows: The upstream primer sequence (from Genbank Accession number (T25352), 5′ GGG GGG CTC GAG AAC AAC ATC ACC ATG ACA (Sequence ID No. 16), and the downstream primer sequences taken from Griffiths et al., (Nature Medicine 3:89-93, 1997) where the two degenerate primers are combined for a degeneracy of 960 sequences, 5′ GGG GGG ATC GAT TCA NAC (G/A/T)AT NGC YCT RAA (Sequence ID No. 17). The abbreviations in the degenerate primers are as follows R is A or G; Y is C or T; and N is A,T,C,G. The resulting 1.4 Kb human hENT1 cDNA, has a Xho I at the 5′ end and Cla I at the 3′ end. It is inserted into the Moloney retroviral vector KT-1 at the Xho I and Cla I sites to make KT1/hENT1. KT1/hENT1 is used to make a VCL by pseudotyping with VSV G protein.

### Example 11

### INTRAVENOUS ADMINISTRATION OF RECOMBINANT RETROVIRUSES EXPRESSING FACTOR VIII

A. Construction of Full-Length and B Domain Deleted Factor Viii cDNA Retroviral Vectors

The following is a description of the construction of several retroviral vectors encoding a full-length factor VIII cDNA. Further discussion is also provided in U.S. application Ser. No. 08/366,851. Due to the packaging constraints of retroviral vectors and because selection for transduced cells is not a requirement for therapy, a retroviral backbone, e.g., KT-1, lacking a selectable marker gene is employed.

1. Production of Plasmid Vectors Encoding Full-Length Factor VIII

A gene encoding full length factor VIII can be obtained from a variety of sources. One such source is the plasmid pCIS-F8 (see EP 0 260 148), which contains a full length factor VIII cDNA whose expression is under the control of a CMV major immediate-early (CMV MIE) promoter and enhancer. The factor VIII cDNA contains approximately 80 bp of 5′ untranslated sequence from the factor VIII gene and a 3′ untranslated region of about 500 bp. In addition, between the CMV promoter and the factor VIII sequence lies a CMV intron sequence, or “cis” element. The cis element, spanning about 280 bp, comprises a splice donor site from the CMV major immediate-early promoter about 140 bp upstream of a splice acceptor from an immunoglobulin gene.

More specifically, a plasmid, designated pJW-2, encoding a retroviral vector for expressing full length factor VIII is constructed using the KT-1 backbone from pKT-1. Briefly, in order to facilitate directional cloning of the factor VIII cDNA insert into pKT-1, the unique Xho I site is converted to a Not I site by site directed mutagenesis. The resultant plasmid vector is then opened with Not I and Cla I. pCIS-F8 is digested to completion with Cla I and Eag I, for which there are two sites, to release the fragment encoding full length factor VIII. This fragment is then ligated into the Not I/Cla I restricted vector to generate a plasmid designated pJW-2.

2. Construction of a Truncated Factor VIII retroviral vector (ND-5)

A plasmid vector encoding a truncation of about 80% (approximately 370 bp) of the 3′ untranslated region of the factor VIII cDNA, designated pND-5, is constructed in a pKT-1 vector as follows: As described for pJW-2, the pKT-1 vector employed has its Xho I restriction site replaced by that for Not I. The factor VIII insert is generated by digesting pCIS-F8 with Cla I and Xba I, the latter enzyme cutting 5′ of the factor VIII stop codon. The approximately 7 kb fragment containing all but the 3′ coding region of the factor VIII gene is then purified. pCIS-F8 is also digested with Xba I and Pst I to release a 121 bp fragment containing the gene's termination codon. This fragment is also purified and then ligated in a three way ligation with the larger fragment encoding the rest of the factor VIII gene and Cla I/Pst I restricted BLUESCRIPT® KS⁺ plasmid (Stratagene, supra) to produce a plasmid designated pND-2.

The unique Sma I site in pND-2 is then changed to a Cla I site by ligating Cla I linkers (New England Biolabs, Beverly, MA) under dilute conditions to the blunt ends created by a Sma I digest. After recircularization and ligation, plasmids containing two Cla I sites are identified and designated pND-3.

The factor VIII sequence in pND-3, bounded by Cla I sites and containing the full length gene with a truncation of much of the 3′ untranslated region, is cloned as follows into a plasmid backbone derived from a Not I/Cla I digest of pKT-1 (a pKT-1 derivative by cutting at the Xho I site, blunting with Klenow, and inserting a Not I linker (New England Biolabs)), which yields a 5.2 kb Not I/Cla I fragment. pCIS-F8 is cleaved with Eag I and Eco RV and the resulting fragment of about 4.2 kb, encoding the 5′ portion of the full length factor VIII gene, is isolated. pND-3 is digested with Eco RV and Cla I and a 3.1 kb fragment is isolated. The two fragments containing portions of the factor VIII gene are then ligated into the Not I/Cla I digested vector backbone to produce a plasmid designated pND-5.

3. Construction of the B-Domain Deleted Vector

The precursor DNA for the B-deleted FVIII is obtained from Miles Laboratory. This expression vector is designated p25D and has the exact backbone as pCISF8 above. The Hpa I site at the 3′ of the FVIII8 cDNA in p25D is modified to Cla-I by oligolinkers. An Acc I to Cla I fragment is clipped out from the modified p25D plasmid. This fragment spans the B-domain deletion and includes the entire 3′ two-thirds of the cDNA. An Acc I to Cla I fragment is removed from the retroviral vector JW-2 above, and replaced with the modified B-domain deleted fragment just described. This is designated B-del-1.

4. Construction of Factor VIII vectors with non-immunogenic markers/PAE genes

The above vectors are all made in the KT-1 backbone that has no selectable marker. They can similarly be constructed in the pBA5 vector backbone (FIG. 3). Briefly a selectable marker can be introduced into them by cutting at a single Cla I site and introducing an expression cassete for the marker as was done for the neomycin marker in Example 1. In this way a cassette expressing any of placental alkaline phosphatase, deoxycytidine kinase, cytochrome P-450 or other suitable non-immunogenic marker/PAE can be introduced. The cassette will have the cDNA linked to a promoter such as the SV40 promoter in Example 1 and no polyadenylation site. Other suitable internal promoters (e.g., CMV from the pCI-PLAP vector in Example 4) can also be utilized. Such vectors are called pJW-2-PLAP, pJW-2-DCK, pND5-PLAP, pBdell-PLAP and pBdell-DCK, etc.

5. Construction of pCF8-PLAP

a. Deletion of the 3′ end of human factor VIII cDNA

A Xba I to Not I fragment was amplified from retroviral vector pCF8 (also designated pMBF8; see PCT Application No. US 97/11785) utilizing the following primers and PCR:

1. FVIII 3′ Xba; (Sequence ID No.31)
5′ GAATGGCAAAGTAAAGGTTTTTCAGGG
(33 bp upstream of the 3′ Xba I)
2. FVIII 3′ Not; (Sequence ID No.32)
5′ ATAGTTAGCGGCCGCAACCCGGGCCACCCTCAGTAGAGGTCCTG

The amplified DNA was digested with Xba I and Not I and cloned into the BlueScript SK⁻ plasmid (Stratagene) which had been digested with Xba I and Not I. The resulting plasmid was named pKS-121.

pCF8 was also digested with PflM I and Not I, and dephosphorylated using CIAP. A 8.3 kb fragment was isolated and gel purified. A 1.3 kb fragment was also isolated from pCF8 by digesting with PflM I and Xba I. A 121 bp fragment was isolated from KS-121 by digesting with Xba I and Not I. All 3 fragments were ligated together to generate the plasmid, pCF8-D3′. This plasmid is similar to pCF8 except that the 3′ non-coding region of the FVIII cDNA has been deleted and a short linker was added.

b. Insertion of the PLAP cDNA

pBAAP (containing PLAP) was digested with Xho I, blunted using T4 DNA Polymerase large fragment (Klenow), and dephosphorylated using CIAP. It was then ligated in the presence of excess Not I linker (Phosphorylated). The resulting plasmid, pBAAP X/N, was digested with Not I and the 1.9 kb fragment (Not I to Not I PLAP cDNA) was ligated into pCF8-D3′ linearized with Not I. The resulting plasmids were analyzed using restriction mapping to determine the orientation of the insert. The resulting plasmid, named pCF8-PLAP, is a dicistronic vector including both cDNAs separated by a short spacer of 59 bp.

B. Assay for Factor VIII Expression

1. Assay of KT-ND5 Vector Expression by Transient Packaging and Transduction of Murine Cells

Cell lines, L33, (Dennert, USC Comprehensive Cancer Center, Los Angeles, Calif., Patek, et. al., Int. J of Cancer 24:624-628, 1979), BC1OME (Patek, et al., Cell Immuno 72:113, 1982, ATCC# TIB85), L33env, and BCenv (L33env and BCenv express HIV-1 IIIBenv, Warner et al, AIDS Res. and Human Retrovirus 7:645, 1991), transduced with the KT-ND5-DCK vector, carrying the amphotropic or VSVG envelope protein are examined for the expression of factor VIII. Non-transduced cells are also analyzed for factor VIII expression and compared with KT-ND5-DCK transduced cells to determine the effect of transduction on protein expression.

Murine cell lines, L33-KT-ND5-DCK, L33env-KT-ND5-DCK, L33env, L33, BC1OME, BClOME-KT-ND5-DCK, BCenv, and BCenv-KT-ND5-DCK, are tested for expression of the KT-ND5-DCK molecule. Cells are grown to subconfluent density and the supernatant is removed following centrifugation at 200 xg. The samples are diluted and assayed by the COATEST® Factor VIII assay (KabiVitrum Diagnostica, Molndal, Sweden).

The assay is performed as follows: 100 μl of culture media sample is mixed with 200 μl of working buffer provided in the kit. The mixture is incubated at 37 C. for 4-5 min., after which 100 μL of a 0.025 M CaCl₂ stock solution is added, followed by a 5 min. 37 C. incubation. 200 μL of the chromogenic reagent (20 mg S-2222, 335 μg synthetic thrombin inhibitor, 1-2581, in 10 mL) is then mixed in. After a 5 min. incubation at 37 C., 100 μL of 20% acetic acid or 2% citric acid is added to stop the reaction. Absorbance is then measured against a blank comprising 50 mM Tris, pH 7.3, and 0.2% bovine serum albumin (BSA). A standard curve based on dilutions of normal human plasma (1.0 IU factor VIII/mL) is used and the assays should be performed in plastic tubes. Serum levels of factor VIII in non-hemophilic patients are in the range of 200 ng/mL.

When this assay is used for patient samples, 9 volumes of blood are mixed with one volume of 0.1 M sodium citrate, at a neutral pH, and centrifuged at 2,000×g for 5-20 min. at 20-25 C. to pellet cells. Due to heat lability of factor VIII, plasma samples should be tested within 30 min. of isolation or stored immediately at −70 C., although as much as 20% of factor VIII activity may be lost during freezing and thawing.

2. Assay of KT-ND5-DCK Vector Expression by Transient Packaging and Transduction of Human Cells

Cell lines transduced with KT-ND5-DCK are examined for expression of factor VIII. Non-transduced cells are analyzed to compare with KT-ND5-DCK transduced cells and determine the effect that transduction has on expression.

Two human cell lines, JY and JY-KT-ND5-DCK are tested for expression of KT-ND5-DCK. Suspension cells grown to 10⁶ cells/ml are removed from culture flasks by pipet and pelleted by centrifugation at 200 xg. The supernatant is removed, diluted, and assayed by the Coatest^{R} Factor VIII assay as described above in Example 2B 1.

C. Transient Transfection and Transduction of Packaging Cell Lines HX and DA with the Vector Construct KT-ND5-DCK

1. Plasmid DNA Transfection

The packaging cell line, HX (WO92/05266), are seeded at 5.0×10⁵ cells on a 10 cm tissue culture dish on day 1 with Dulbecco's Modified Eagle Medium (DMEM) and 10% fetal bovine serum (FBS). On day 2, the media is replaced with 5.0 ml fresh media 4 hours prior to transfection. A standard calcium phosphate-DNA co-precipitation is performed by mixing 40.0 μl 2.5 M CaCl₂, 10 μg plasmid DNA, and deionized H₂O to a total volume of 400 μl. Four hundred microliters of the DNA-CaCl₂ solution is added dropwise with constant agitation to 400 μl precipitation buffer (50 mM HEPES-NaOH, pH 7.1; 0.25 M NaCl and 1.5 mM Na₂HPO₄—NaH₂PO₄). This mixture is incubated at room temperature for 10 minutes. The resultant fine precipitate is added to a culture dish of cells. The cells are incubated with the DNA precipitate overnight at 37° C. On day 3, the media is aspirated and fresh media is added. The supernatant is removed on day 4, passed through a 0.45 μl filter, and stored at −80° C.

Alternatively, 29 2 3 cells (WO 92/05266) (these are 293 cells expressing gag and pol) are transfected with the vector DNA and the plasmid pMLP-VSVG (or other VSVG encoding plasmids) to yield VSVG psuedotyped vector particles that are harvested and stored as described above.

2. Packaging Cell Line Transduction

DA (an amphotropic cell line derived from a D17 cell line ATCC No. 183, WO 92/05266) cells are seeded at 5.0×10⁵ cells/10 cm tissue culture dish in 10 ml DMEM and 10% FBS, 4 μg/ml polybrene (Sigma, St. Louis, MO) on day 1. On day 2, 3.0 ml, 1.0 ml and 0.2 ml of the freshly collected virus-containing HX media is added to the cells. The cells are incubated with the virus overnight at 37° C., followed by cloning by limiting dilution, and the best clones are selected by, e.g., PCR titering or Fast Red staining as described in Example 5.

Using these procedures, cell lines are derived that produce greater than or equal to 10⁶ cfu/ml in culture.

The packaging cell line HX can be transduced with vector generated from the DA vector producing cell line in the same manner as described for transduction of the DA cells from HX supernatant.

3. Generation of Producer Cell Line via One Packaging Cell Line

In some situations it may be desirable to avoid using more than one cell line in the process of generating producer lines. In this case, DA cells are seeded at 5.0×10⁵ cells on a 10 cm tissue culture dish on day 1 with DMEM and 10% irradiated (2.5 megarads minimum) FBS. On day 2, the media is replaced with 5.0 ml fresh media 4 hours prior to transfection. A standard calcium phosphate-DNA coprecipitation is performed by mixing 60 μl 2.0 M CaCl₂, 10 μg MLP-G plasmid, 10 μg KT-ND5-DCK retroviral vector plasmid, and deionized water to a volume of 400 μl. Four hundred microliters of the DNA-CaCl₂ solution is added dropwise with constant agitation to 400 μl 2X precipitation buffer (50 mM HEPES-NaOH, pH 7.1, 0.25 M NaCl and 1.5 mM Na₂HPO₄-NaH₂PO₄). This mixture is incubated at room temperature for 10 minutes. The resultant fine precipitate is added to a culture dish of DA cells plated the previous day. The cells are incubated with the DNA precipitate overnight at 37° C., followed by cloning by limiting dilution. The best clones are selected by, e.g., PCR titering or Fast Red staining as described in Example 5.

D. Detection of Replication Competent Retroviruses (RCR)

1. The Extended S⁺L⁻ Assay

The extended S⁺L⁻ assay determines whether replication competent, infectious virus is present in the supernatant of the cell line of interest. The assay is based on the empirical observation that infectious retroviruses generate foci on the indicator cell line MiCl₁ (ATCC No. CCL 64.1). The MiCl₁ cell line is derived from the Mv1Lu mink cell line (ATCC No. CCL 64) by transduction with Murine Sarcoma Virus (MSV). It is a non-producer, non-transformed, revertant clone containing a replication defective murine sarcoma provirus, S⁺, but not a replication competent murine leukemia provirus, L⁻. Infection of MiCl₁ cells with replication competent retrovirus “activates” the MSV genome to trigger “transformation” which results in foci formation.

Supernatant is removed from the cell line to be tested for presence of replication competent retrovirus and passed through a 0.45 μ filter to remove any cells. On day 1, Mv1Lu cells are seeded at 1.0×10⁵ cells per well (one well per sample to be tested) of a 6 well plate in 2 ml DMEM, 10% FBS and 8 μg/ml polybrene. MvlLu cells are plated in the same manner for positive and negative controls on separate 6 well plates. The cells are incubated overnight at 37° C., 10% CO₂. On day 2, 1.0 ml of test supernatant is added to the Mv1Lu cells. The negative control plates are incubated with 1.0 ml of media. The positive control consists of three dilutions (200 focus forming units (ffu), 20 ffu and 2 ffu each in 1.0 ml media) of MA virus (referred to as pAM in Miller et al., Molec. and Cell Biol. 5:431, 1985) which is added to the cells in the positive control wells. The cells are incubated overnight. On day 3, the media is aspirated and 3.0 ml of fresh DMEM and 10% FBS is added to the cells. The cells are allowed to grow to confluency and are split 1:10 on day 6 and day 10, amplifying any replication competent retrovirus. On day 13, the media on the Mv1Lu cells is aspirated and 2.0 ml DMEM and 10% FBS is added to the cells. In addition, the MiCl₁ cells are seeded at 1.0×10⁵ cells per well in 2.0 ml DMEM, 10% FBS and 8 μg/ml polybrene. On day 14, the supernatant from the Mv1Lu cells is transferred to the corresponding well of the MiCl₁ cells and incubated overnight at 37° C., 10% CO₂. On day 15, the media is aspirated and 3.0 ml of fresh DMEM and 10% FBS is added to the cells. On day 21, the cells are examined for focus formation (appearing as clustered, refractile cells that overgrow the monolayer and remain attached) on the monolayer of cells. The test article is determined to be contaminated with replication competent retrovirus if foci appear on the MiCl₁ cells. Using these procedures, it can be shown that the HBV core producer cell lines are not contaminated with replication competent retroviruses.

2. Cocultivation of Producer Lines and MdH Marker Rescue Assay

As an alternate method to test for the presence of RCR in a vector-producing cell line, producer cells are cocultivated with an equivalent number of Mus dunni (NIH NIAID Bethesda, MD) cells. Small scale cocultivations are performed by mixing of 5.0×10⁵ Mus dunni cells with 5.0×10⁵ producer cells and seeding the mixture into 10 cm plates (10 ml standard culture media/plate, 4 μg/ml polybrene) at day 0. Every 3-4 days the cultures are split at a 1:10 ratio and 5.0×10⁵ Mus dunni cells are added to each culture plate to effectively dilute out the producer cell line and provide maximum amplifcation of RCR. On day 14, culture supernatants are harvested, passed through a 0.45 μ cellulose-acetate filter, and tested in the MdH marker rescue assay. Large scale cocultivations are performed by seeding a mixture of 1.0×10⁸ Mus dunni cells and 1.0×10⁸ producer cells into a total of twenty T-150 flasks (30 ml standard culture media/flask, 4 μg/ml polybrene). Cultures are split at a ratio of 1:10 on days 3, 6, and 13 and at a ratio of 1:20 on day 9. On day 15, the final supernatants are harvested, filtered and a portion of each is tested in the MdH marker rescue assay.

The MdH marker rescue cell line is cloned from a pool of Mus dunni cells transduced with LHL, a retroviral vector encoding the hygromycin B resistance gene (Palmer et al., PNAS 84:1055-1059, 1987). The retroviral vector can be rescued from MdH cells upon infection of the cells with RCR. One ml of test sample is added to a well of a 6-well plate containing 10⁵ MdH cells in 2 ml standard culture medium (DMEM with 10% FBS, 1% 200 mM L-glutamine, 1% non-essential amino acids) containing 4 μg/ml polybrene. Media is replaced after 24 hours with standard culture medium without polybrene. Two days later, the entire volume of MdH culture supernatant is passed through a 0.45 μ cellulose-acetate filter and transferred to a well of a 6-well plate containing 5.0×10⁴ Mus dunni target cells in 2 ml standard culture medium containing polybrene. After 24 hours, supernatants are replaced with standard culture media containing 250 μg/ml of hygromycin B and subsequently replaced on days 2 and 5 with media containing 200 μg/ml of hygromycin B. Colonies resistant to hygromycin B appear and are visualized on day 9 post-selection, by staining with 0.2% Coomassie blue.

F. Transduction of Human Cells with KT-ND5-DCK Vector Construct

On day one, HT1080 cells are set up at 2×10⁴ cells per well in six well tissue culture plates containing 2 mls standard growth media (DME+10% FBS). On day two, ND-5 FVIII retroviral vector particles from a confluent vector producing cell line are harvested as a HX-ND-5 clone. They are filtered through 0.45 μm syringe filters prior to testing the supernatants. (Alternatively the filtered media supernatants may be frozen at 80 in aliquots for later use.) Polybrene is added to each well such that the final concentration is 8 μg per ml. Thirty minutes later, either diluted or undiluted retroviral vector supernatant is added to duplicate wells. Typical volumes and dilutions are 0.5 ml per well and four or more 1:3 serial dilutions in growth media. As a control, two wells are transduced with the same volume of growth media only. On day three, the wells are refeed with 2 mls of fresh media and the cells allowed to reach confluence, which may typically be about day four or five. On this day, the cells are again refeed with one ml per well fresh growth media. Twenty four hours later the media is harvested and filtered as above.

G. Expression of Transduced Vector For FVIII

The expression of vector transduced human cells for FVIII is detected by the CoatestR assay as described above in Example 2B 1. Activity is assayed relative to supernatant from the control wells by counting the cells per well from the two control wells and normalizing FVIII expression data per 1×10⁶ cells per 24 hours.

H. Administration of Vector Construct

1. Animal Administration Protocol

The intestinal epithelium is an attractive site for gene delivery due to its rapidly proliferating tissue mass and the known location of stem cells in the crypts of Lieberkuhn. The deep location of the stem cells in the crypts and the protective role of the mucus gel layer, makes the retrovirus relatively inaccessible to the tissue cells. However, the accessibility of the retroviral vector to these stem cells can be improved in animal models by the in vivo mucus removal method of Sandberg, J., et al.,(Human Gene Therapy 5:3232-329, 1994).

Male Sprague-Dawley rats obtained from Charles River Breeding Laboratories (Portage, Md.) are anesthetized and the cecum is identified upon opening the peritoneal cavity. A 3 cm ileal segment is isolated from the last Peyer's patch in the terminal ileum and ligated at each end. A plastic catheter attached to a syringe is inserted into the segment and two milliliters of the mucolytic agents dithiothreitol and N-acetyl-cysteine is instilled under mild pressure for two minutes, then removed. This procedure is repeated once again before filling the segment with 0.2 to 2.0 ml of retroviral vector particles at 10⁶ to 10¹⁰ cfu/ml. The ligatures are removed 1 to 4 hours later and the abdominal cavity is sutured. Control animals are instilled with formulation buffer only.

Blood is collected from the tail vein and assayed for factor VIII production by a sandwich ELISA specific for human factor VIII (according to the modified procedure of Zatloukal, K., et al., PNAS 91:5148-5152, 1994). The ELISA is based on two Diagnostica). ESH 4 (25 μg/ml in 1.0 M NaHCO₃/0.5 M NaCl, pH 9.0) is coupled to the ELISA plates overnight at 4° C., washed with 0.1% Tween 20 in PBS, and blocked with 1% BSA in PBS. The samples are applied in 0.05 M Tris-HCl/1 M NaCI/2% BSA, pH 7.5, over 4 hr at room temperature, the plates are washed, and ESH 8 (2.5 μg/ml in 0.05 M Tris-HCl/l M NaCl/2% BSA, pH 7.5,) which has been biotinylated with N-hydroxysuccinimidobiotin (Pierce, Rockford, Ill.) is added for 2 hr at room temperature. The color reaction is performed with peroxidase-conjugated streptavidin (Boehringer Mannheim, Indianapolis, Ind.) and o- phenylenediamine dihydrochloride as substrate. The human factor VIII:c standard (from the National Institute for Biological Standards and Control, Hertfordshire, U.K.) and normal rat plasma are used as references.

2. Human Administration Protocol

Lyophilized recombinant retrovirus containing the gene for Factor VIII expression is formulated into an enteric coated tablet or gel capsule according to known methods in the art. These are described in the following patents: U.S. Pat. No. 4,853,230, EP 225,189, AU 9,224,296, AU 9,230,801, and WO 92144,52.

The capsule is administered orally to be targeted to the jejunum. At 1 to 4 days following oral administration of the recombinant retrovirus, expression of Factor VIII is measured in the plasma and blood by the CoatestR Factor VIII assay.

### Example 12

### PREPARATION OF RECOMBINANT RETROVIRUS FOR DELIVERY OF HUMAN GROWTH HORMONE

A. Preparation of hGH containing Vectors

Vector pDHF828 containing the full-length human growth hormone gene is constructed essentially as follows. Briefly, plasmid pDHF811, was constructed by removing the XhoI-ClaI fragment of the KT-1 retroviral vector described above, and inserting the following oligonucleotide linkers by ligation of the cohesive ends:

Linker sequences:
(SEQUENCE ID# 18) 5′ TCGAGGATCC GCCCGGGCGG
CCGCATCGAT GTCGACG 3′
(SEQUENCE ID# 19) 5′ CGCGTCGA CATCGATGCG
GCCGCCCGGG CGGATCC 3′

In particular, the linkers were annealed at 65° C. for 20 minutes, 42° C. for 20 minutes, 37° C. for 20 minutes, and room temperature for 2 hours. The concentrations of both oligonucleotides was 18 mM and the salt concentration was 100 mM NaCl. After annealing, 50 ml of 1.8 mM annealed linker was digested with ClaI overnight to generate ClaI ends. For ligation, 3nM of KT-1 XhoI-ClaI fragment was mixed with 90nM of linker, and the resultant mixture incubated at 15° C. for 3 hours. The ligated DNA sample was transformed into DH-5α competent cells, followed by screening of transformants.

Plasmid chGH 800 containing the full length cDNA of the hGH gene (Martial, R. A. et al., Science 205:602, 1979) was digested with Hind III, blunt-ended with the Klenow fragment enzyme, and cloned into the Srfl site of pDHF8 11. The resultant plasmid was designated pDHF828.

The above vector is made in the KT-1 backbone that has no selectable marker. It can similarly be constructed in the pBA5 vector backbone (FIG. 3). Briefly a selectable marker is introduced into it by cutting at a single Cla I site and introducing an expression cassete for the marker as was done for the neomycin marker in Example 1. In this way a cassette expressing any of placental alkaline phosphatase, deoxycytidine kinase, cytochrome P-450 or other suitable non-immunogenic marker/PAE can be introduced. The cassette has the cDNA linked to a promoter such as the SV40 promoter in Example 1 and no polyadenylation site. Other suitable internal promoters (e.g., CMV from the pCI-PLAP vector in Example 4) can also be utilized. Such vectors are called pDHF828-PLAP and pDHF828-DCK.

B. Preparation of hGH Expressing Recombinant Retrovirus

The pDHF828-DCK plasmid was then introduced into the HX packaging cell, using standard procedures and assayed using the HGH Chemiluminescence Kit (HGH 1OOT) (Nichols Institute, San Juan Capistrano, CA.), according to a preferred modification of the kit protocol. On day 1, the kit components were warmed to room temperature and gently mixed by inversion before opening any vials. Test samples were centrifuged for 5′ at top speed in a microfuge before using them in order to remove fibrin and other debris. All samples were measured in quadruplicate, including the standards. The incubations are performed in 12×17 polypropylene tubes that have been stored in the dark. One hundred fifty ul of sample or standard were aliquoted into each tube and ul of antibody is added and the samples were mixed gently. One bead was added to each well using the forceps provided in the kit. The tubes were capped, covered with foil, and shaken on an orbital shaker for 24 hr at room temperature. Standards contain 530 pg/ml (STD D), and serial dilutions were made in zero standard of StdD of250, 100,50,25, 10,5, and 2.5 pg/ml.

After 24 hours, the tubes were uncapped and 0.5 ml of wash buffer were added. These wash solution was added with enough force to make the bead bounce up off the bottom of the tube. The samples were washed three times with 2.0 ml nanopure water, and aspirated completely each time. The luminometer determinations were done in 12×75 polycarbonate (clear plastic) tubes stored in the dark. The luminometer was pretested with performance control standards.

Using this assay, HX/HGH-DCK retroviral vector producing cell lines 6 were generated with titers of 4.8×10 cfu/ml. Introduction of the plasmid into DX packaging cells resulted in production of clonal producer cells with a titer of 1.6×10⁷ cfu/ml.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

We claim:

1. A method of delivering a gene delivery vehicle to a warm-blooded animal, comprising administering to a warm-blooded animal a gene delivery vehicle which directs the expression of a non-immunogenic selectable marker.

2. A method of delivering a gene delivery vehicle to a warm-blooded animal, comprising administering to a warm-blooded animal a gene delivery vehicle which directs the expression of a non-immunogenic molecule which is capable of activating an otherwise inactive compound into an active compound.

3. The method according to claims 1 or 2 wherein said vector construct also directs the expression of a selected heterologous nucleic acid seqeunce.

4. The method according to claim 1 wherin said selectable marker is selected from the group consisting of alkaline phosphatase, α-Galactosidase, β-glucosidase, β-glucuronidase, Carboxypeptidase A, Cytochrome P450, γ-glutamyl transferase; reductases such as Azoreductase, DT diaphorase and Nitroreductase; and oxidases such as glucose oxidase and xanthine oxidase.

5. The method according to claim 1 wherin said compound capable of activating an otherwise inactive compound into an active compound is selected from the group consisting of alkaline phosphatase, α-Galactosidase, β-glucosidase, β-glucuronidase, Carboxypeptidase A, Cytochrome P450, γ-glutamyl transferase; reductases such as Azoreductase, DT diaphorase and Nitroreductase; and oxidases such as glucose oxidase and xanthine oxidase.

6. The method according to any one of claim 1 or 2 wherein said gene delivery vehicle is a retroviral vector construct.

7. The method according to any one of claim 1 or 2 wherein said gene delivery vehicle is selected from the group consisting of poliovirus vectors, rhinovirus vectors, pox virus vectors, canary pox virus vectors, vaccinia virus vectors, influenza virus vectors, adenovirus vectors, parvovirus vectors, adeno-associated viral vectors, herpesvirus vectors, SV 40 vectors, lenti virus vectors, measles virus vectors, astrovirus vectors, corona virus vectors and Alphavirus vectors.

8. The method according to any one of claim 1 or 2 wherein said gene delivery vehicle is selected from the group consisting of polycation condensed nucleic acids, liposome entrapped nucleic acids, naked DNA or RNA and producer cell lines.

9. The method according to claim 3 wherein said heterologous sequence is a gene encoding a cytotoxic protein.

10. The method according to claim 9 wherein said cytotoxic protein is selected from the group consisting of ricin, abrin, diphtheria toxin, cholera toxin, gelonin, pokeweed, antiviral protein, tritin, Shigella toxin and Pseudomonas exotoxin A.

11. The method according to claim 3 wherein said heterologous sequence is an antisense sequence.

12. The method according to claim 3 wherein said heterologous sequence encodes an immune accessory molecule.

13. The method according to claim 12 wherein said immune accessory molecule is selected from the group consisting of α interferon, βinterferon, IL-1, IL-3, IL-4, IL-5, IL-6, IL-7, IL-8, IL-9, IL-10, IL-11I and IL-13.

14. The method according to claim 12 wherein said immune accessory molecule is selected from the group consisting of IL-2, IL-12 and gamma-interferon.

15. The method according to claim 12 wherein said immune accessory molecule is selected from the group consisting of ICAM-1, ICAM-2, β-microglobin, LFA3, and HLA class I and HLA class II molecules.

16. The method according to claim 3 wherein said heterologous sequence is a ribozyme.

17. The method according to claim 3 wherein said heterologous sequence is a replacement gene.

18. The method according to claim 17 wherein said replacement gene encodes a protein selected from the group consisting of Factor VIII, ADA, HPRT, CFTCR and the LDL Receptor.

19. The method according to claim 3 wherein said heterologous sequence encodes an immunogenic portion of a virus selected from the group consisting of HBV, HCV, HPV, EBV, FeLV, FIV and HIV.

20. The method according to claims 1 or 2 wherein said gene delivery vehicle is introduced into cells ex vivo, followed by administration of said gene delivery vehicle containing cells to said warm-blooded animal.
